SEQUENCE LISTING (86 sequences)

SEQ ID NO: 1 - DNA, 1688 bp, Homo sapiens
ggagtcgaccgctcgggcagcgccaccgccacgagagcccgggacgcgggaaagaccgaaaggaagaggaagaggcaccggtggccatggggctggaggcggcgcgcgagctggagtgcgcggcgctgggcacgctgctgcgggatccgcgggaggcggaacgcacgctgctgctggactgccgccccttcctggccttctgccggcgccacgtgcgcgccgcgcggccagtgccttggaacgcgctgctgcggcgccgcgcgcgcggccctcctgccgccgttctcgcctgcctgctgcccgaccgcgcgctgcggacgcgcctggtccgcggggagctggcgcgggccgtggtgctggacgagggcagtgcctcggtggcggagctccggcccgacagcccggctcatgtgctgctggccgcgctgctgcacgagacccgcgcggggcccactgccgtgtacttcctgcgaggaggcttcgacggcttccagggctgctgtcccgatctgtgctctgaggcccccgcccctgcgctgccgccaacaggggacaaaaccagccgctccgactccagggctcctgtctacgaccagggtggccctgtggagatcttgccctacctgttcctgggcagctgcagtcactcgtcagacctgcaggggctgcaggcctgtggcatcacagccgtcctcaacgtgtccgccagctgccccaaccactttgagggccttttccgctacaagagtatccctgtggaggacaaccagatggtggagatcagtgcctggttccaggaggccataggcttcattgactgggtgaagaacagcggaggccgggtgctggtgcactgccaggcgggtatctcgcgctctgccaccatctgtctggcatacctcatgcagagtcgccgtgtgcggctggacgaggcctttgacttcgttaagcagcgccggggggtcatctcccccaacttcagtttcatggggcagctgctgcagtttgagacccaggtgctgtgtcactgaggtggtgcccctctgcctgcctgccccactgtgctggcaggagctgactgtggactggtgggctcccctctgggccagcacagtcccctcacctctggcagggctgctacctcctcagagtttcagaagcccccacatgggggctctaggaatgccggcatgctggtctttccgacctggtgctcttctgctgggggactgaggctggccctcattcggggtcgggaaccaagggtgtgtctgctctttccctccccatcctctggcagaaatcagctagacgctataccgtggactctccctggtccaccaccatgttgaagcccttggcagcctgagagctccaaggaacaagctgtgacaaccaggagccctgtctgtgggttcgtctgcccagggcctggagcccaagccctgtgttcctggggaagctggggacttgggaagtgatgggtgtgtcatgttgcgtgtgtctgtctgtgagcctttcacacctgtgctggcgctggaaaattatttgtgctcagctgacatttaacactccctcccccgcttcctcctagccctgtgggcaggggttggaaacttagcactttatatttatacagaacattcaggatatgtcaataaaatattgttatatttaaaaaacaacaa

SEQ ID NO: 2 - protein, 314 aa, Homo sapiens
MGLEAARELECAALGTLLRDPREAERTLLLDCRPFLAFCRRHVRAARPVPWNALLRRRARGPPAAVLACLLPDRALRTRLVRGELARAVVLDEGSASVAELRPDSPAHVLLAALLHETRAGPTAVYFLRGGFDGFQGCCPDLCSEAPAPALPPTGDKTSRSDSRAPVYDQGGPVEILPYLFLGSCSHSSDLQGLQACGITAVLNVSASCPNHFEGLFRYKSIPVEDNQMVEISAWFQEAIGFIDWVKNSGGRVLVHCQAGISRSATICLAYLMQSRRVRLDEAFDFVKQRRGVISPNFSFMGQLLQFETQVLCH

SEQ ID NO: 3 - DNA, 1516 bp, Mus musculus
agggagaggacatccggaagcaggaggctttgcgatgccgatcgccatggggctggagacggcgtgcgagctagagtgcgcggcgctgggcgcgctgctgcgggagccgcgggaggccgagcgcacgctgctgctcgactgccgcccgttcctggccttctgccggagccacgtgcgcgccgcgcgaccggtgccctggaacgcgctgctgcggcggcgtgcgcgcggcacccccgccgctgccctcgcctgtctgttgcccgaccgcgcactccgggcgcgcctgggccgcggggagctggcccgtgccgtggtgctggatgaaagcagcgcgtctgtggccgagctcccgccggacggccccgctcacctgctgctagccgcactgcagcacgagatgcgcgggggacccacgaccgtgtgcttcttgcgaggcggtttcaaaagcttccagacgtactgtccggatctgtgctctgaagctcctgcccaggccctgcctcctgctggggccgaaaatagcaactctgacccgagggttccgatctatgaccagggtggtcctgtggaaatcttgccctacctgtacttgggcagctgcaaccactcctcagatcttcagggcctgcaggcctgcggcatcacagcagttctcaatgtctctgccagctgccccaaccactttgagggtcttttccattacaagagcattccagtagaagataaccagatggtggagataagtgcctggttccaggaggctatcagcttcatagactcggtgaagaatagtgggggccgggtgctggtgcactgccaagctggcatctctcgctcagccaccatctgcctggcatacctgattcagagccaccgggtacggctggacgaggcctttgactttgttaagcaacgtcggggagtcatctcccccaacttcagtttcatggggcagctgctacagctggagacccaggtgctgtgtcactgaggcaaagcccagcctggccccacccacagctctggctttgactgactcctgggggaagctgctgtggcccagctcctccctcctcatcttgccgtcccactggggtgctaggaaatccaggatgacggctgctctgatatggtgctcttctgaggtggcataagggctggcccttatttgctctccctccttcgacttgcagaaattaatttaattaattaatttactatattaaagcccttggtcacccaagggctcagaaaacaagctgtgacaagcagaaaccatgtctaggggtgtgcagcccttgacccaaagcttaagccttgtgctcccaggggagccaggaagtgacgtgtgtgtcatgttacggacatctgacttttgtgtgtgtgtgtgtgggcatctcgctgtaattggtgctgaaaagttatttgtgttcaactgacatttaacgctctctccccaacttcctcccggccctgtgggccagggaggggcgttggaaacagcactttatattta

SEQ ID NO: 4 - protein, 318 aa, Mus musculus
MPIAMGLETACELECAALGALLREPREAERTLLLDCRPFLAFCRSHVRAARPVPWNALLRRRARGTPAAALACLLPDRALRARLGRGELARAVVLDESSASVAELPPDGPAHLLLAALQHEMRGGPTTVCFLRGGFKSFQTYCPDLCSEAPAQALPPAGAENSNSDPRVPIYDQGGPVEILPYLYLGSCNHSSDLQGLQACGITAVLNVSASCPNHFEGLFHYKSIPVEDNQMVEISAWFQEAISFIDSVKNSGGRVLVHCQAGISRSATICLAYLIQSHRVRLDEAFDFVKQRRGVISPNFSFMGQLLQLETQVLCH

SEQ ID NO: 5 - DNA, 21 bp, Artificial sequence
  siRNA sense strand oligonucleotide
agaccgaaaggaagaggaatt

SEQ ID NO: 6 - DNA, 21 bp, Artificial sequence
  siRNA sense strand oligonucleotide
aggaagaggaagaggcacctt

SEQ ID NO: 7 - DNA, 21 bp, Artificial sequence
  siRNA sense strand oligonucleotide
  misc_feature (17)..(17): n=uracil
gaggaagaggcaccggnggtt

SEQ ID NO: 8 - DNA, 21 bp, Artificial sequence
  siRNA sense strand oligonucleotide
  misc_feature (11)..(11): n=uracil
  misc_feature (17)..(17): n=uracil
gaggcaccggnggccanggtt

SEQ ID NO: 9 - DNA, 21 bp, Artificial sequence
  siRNA sense strand oligonucleotide
  misc_feature (8)..(8): n=uracil
  misc_feature (11)..(11): n=uracil
  misc_feature (14)..(14): n=uracil
  misc_feature (17)..(17): n=uracil
  misc_feature (19)..(19): n is a, c, g, or t
cgcacgcngcngcnggacntt

SEQ ID NO: 10 - DNA, 21 bp, Artificial sequence
  siRNA sense strand oligonucleotide
  misc_feature (6)..(6): n=uracil
  misc_feature (9)..(9): n=uracil
cgcgcngcngcggcgccgctt

SEQ ID NO: 11 - DNA, 21 bp, Artificial sequence
  siRNA sense strand oligonucleotide
  misc_feature (9)..(9): n=uracil
  misc_feature (15)..(15): n=uracil
ccagccgcnccgacnccagtt

SEQ ID NO: 12 - DNA, 21 bp, Artificial sequence
  siRNA sense strand oligonucleotide
  misc_feature (3)..(3): n=uracil
  misc_feature (5)..(5): n=uracil
  misc_feature (14)..(14): n=uracil
cgngnccgccagcngcccctt

SEQ ID NO: 13 - DNA, 21 bp, Artificial sequence
  siRNA sense strand oligonucleotide
  misc_feature (4)..(4): n=uracil
  misc_feature (6)..(6): n=uracil
  misc_feature (10)..(10): n=uracil
  misc_feature (12)..(12): n=uracil
gagnancccngnggaggactt

SEQ ID NO: 14 - DNA, 21 bp, Artificial sequence
  siRNA sense strand oligonucleotide
  misc_feature (6)..(6): n=uracil
  misc_feature (9)..(9): n=uracil
  misc_feature (15)..(15): n=uracil
  misc_feature (19)..(19): n=uracil
ccaganggnggagancagntt

SEQ ID NO: 15 - DNA, 21 bp, Artificial sequence
  siRNA sense strand oligonucleotide
  misc_feature (18)..(18): n=uracil
gaacagcggaggccgggngtt

SEQ ID NO: 16 - DNA, 21 bp, Artificial sequence
  siRNA sense strand oligonucleotide
  misc_feature (15)..(15): n=uracil
  misc_feature (18)..(18): n=uracil
cagcggaggccgggngcngtt

SEQ ID NO: 17 - DNA, 21 bp, Artificial sequence
  siRNA sense strand oligonucleotide
  misc_feature (15)..(15): n=uracil
  misc_feature (18)..(18): n=uracil
gcagcgccggggggncanctt

SEQ ID NO: 18 - DNA, 21 bp, Artificial sequence
  siRNA sense strand oligonucleotide
  misc_feature (2)..(3): n=uracil
  misc_feature (7)..(9): n=uracil
  misc_feature (12)..(12): n=uracil
cnncagnnncanggggcagtt

SEQ ID NO: 19 - DNA, 21 bp, Artificial sequence
  siRNA sense strand oligonucleotide
  misc_feature (10)..(10): n=uracil
  misc_feature (17)..(17): n=uracil
  misc_feature (19)..(19): n=uracil
gcccccacangggggcncntt

SEQ ID NO: 20 - DNA, 21 bp, Artificial sequence
  siRNA sense strand oligonucleotide
  misc_feature (1)..(1): n=uracil
  misc_feature (9)..(9): n=uracil
  misc_feature (12)..(12): n=uracil
  misc_feature (15)..(15): n=uracil
  misc_feature (17)..(19): n=uracil
ngccggcangcnggncnnntt

SEQ ID NO: 21 - DNA, 21 bp, Artificial sequence
  siRNA sense strand oligonucleotide
  misc_feature (8)..(8): n=uracil
  misc_feature (10)..(10): n=uracil
  misc_feature (12)..(12): n=uracil
  misc_feature (14)..(14): n=uracil
  misc_feature (17)..(17): n=uracil
  misc_feature (19)..(19): n=uracil
ccaagggngngncngcncntt

SEQ ID NO: 22 - DNA, 21 bp, Artificial sequence
  siRNA sense strand oligonucleotide
  misc_feature (4)..(4): n=uracil
  misc_feature (6)..(6): n=uracil
  misc_feature (8)..(8): n=uracil
  misc_feature (10)..(10): n=uracil
  misc_feature (13)..(13): n=uracil
  misc_feature (15)..(17): n=uracil
gggngngncngcncnnncctt

SEQ ID NO: 23 - DNA, 21 bp, Artificial sequence
  siRNA sense strand oligonucleotide
  misc_feature (2)..(2): n=uracil
  misc_feature (7)..(7): n=uracil
  misc_feature (14)..(14): n=uracil
  misc_feature (16)..(16): n=uracil
ancagcnagacgcnanacctt

SEQ ID NO: 24 - DNA, 21 bp, Artificial sequence
  siRNA sense strand oligonucleotide
  misc_feature (5)..(6): n=uracil
  misc_feature (14)..(14): n=uracil
gcccnnggcagccngagagtt

SEQ ID NO: 25 - DNA, 21 bp, Artificial sequence
  iRNA sense strand oligonucleotide
  misc_feature (10)..(10): n=uracil
  misc_feature (12)..(12): n=uracil
ggaacaagcngngacaacctt

SEQ ID NO: 26 - DNA, 21 bp, Artificial sequence
  siRNA sense strand oligonucleotide
  misc_feature (6)..(6): n=uracil
  misc_feature (8)..(8): n=uracil
caagcngngacaaccaggatt

SEQ ID NO: 27 - DNA, 21 bp, Artificial sequence
  siRNA sense strand oligonucleotide
  misc_feature (3)..(3): n=uracil
  misc_feature (5)..(5): n=uracil
gcngngacaaccaggagcctt

SEQ ID NO: 28 - DNA, 21 bp, Artificial sequence
  siRNA sense strand oligonucleotide
  misc_feature (11)..(11): n=uracil
  misc_feature (13)..(13): n=uracil
  misc_feature (15)..(15): n=uracil
  misc_feature (17)..(17): n=uracil
ccaggagcccngncngnggtt

SEQ ID NO: 29 - DNA, 21 bp, Artificial sequence
  siRNA sense strand oligonucleotide
  misc_feature (5)..(5): n=uracil
  misc_feature (7)..(7): n=uracil
  misc_feature (9)..(10): n=uracil
  misc_feature (13)..(13): n=uracil
gcccngngnnccnggggaatt

SEQ ID NO: 30 - DNA, 21 bp, Artificial sequence
  siRNA sense strand oligonucleotide
  misc_feature (3)..(3): n=uracil
  misc_feature (10)..(11): n=uracil
  misc_feature (18)..(18): n=uracil
gcnggggacnngggaagngtt

SEQ ID NO: 31 - DNA, 21 bp, Artificial sequence
  siRNA sense strand oligonucleotide
  misc_feature (2)..(2): n=uracil
  misc_feature (5)..(5): n=uracil
  misc_feature (9)..(9): n=uracil
  misc_feature (11)..(11): n=uracil
  misc_feature (13)..(13): n=uracil
  misc_feature (16)..(16): n=uracil
  misc_feature (18)..(19): n=uracil
gngangggngngncangnntt

SEQ ID NO: 32 - DNA, 21 bp, Artificial sequence
  siRNA sense strand oligonucleotide
  misc_feature (1)..(2): n=uracil
  misc_feature (4)..(6): n=uracil
  misc_feature (8)..(8): n=uracil
  misc_feature (11)..(11): n=uracil
  misc_feature (16)..(16): n=uracil
nnannngngcncagcngactt

SEQ ID NO: 33 - DNA, 21 bp, Artificial sequence
  siRNA sense strand oligonucleotide
  misc_feature (4)..(4): n=uracil
  misc_feature (8)..(8): n=uracil
  misc_feature (16)..(17): n=uracil
cacncccncccccgcnncctt

SEQ ID NO: 34 - DNA, 21 bp, Artificial sequence
  siRNA sense strand oligonucleotide
  misc_feature (3)..(4): n=uracil
  misc_feature (10)..(12): n=uracil
  misc_feature (14)..(14): n=uracil
  misc_feature (16)..(18): n=uracil
acnnagcacnnnanannnatt

SEQ ID NO: 35 - DNA, 21 bp, Artificial sequence
  siRNA sense strand oligonucleotide
  misc_feature (3)..(4): n=uracil
  misc_feature (10)..(10): n=uracil
  misc_feature (12)..(12): n=uracil
  misc_feature (14)..(14): n=uracil
  misc_feature (18)..(18): n=uracil
canncagganangncaanatt

SEQ ID NO: 36 - DNA, 21 bp, Artificial sequence
  siRNA antisense strand oligonucleotide
  misc_feature (1)..(2): n=uracil
  misc_feature (5)..(5): n=uracil
  misc_feature (7)..(8): n=uracil
  misc_feature (11)..(13): n=uracil
  misc_feature (17)..(17): n=uracil
  misc_feature (19)..(19): n=uracil
nnccncnnccnnncggncntt

SEQ ID NO: 37 - DNA, 21 bp, Artificial sequence
  siRNA antisense strand oligonucleotide
  misc_feature (3)..(3): n=uracil
  misc_feature (7)..(7): n=uracil
  misc_feature (9)..(10): n=uracil
  misc_feature (13)..(13): n=uracil
  misc_feature (15)..(16): n=uracil
  misc_feature (19)..(19): n=uracil
ggngccncnnccncnnccntt

SEQ ID NO: 38 - DNA, 21 bp, Artificial sequence
  siRNA antisense strand oligonucleotide
  misc_feature (8)..(8): n=uracil
  misc_feature (12)..(12): n=uracil
  misc_feature (14)..(15): n=uracil
  misc_feature (18)..(18): n=uracil
ccaccggngccncnnccnctt

SEQ ID NO: 39 - DNA, 21 bp, Artificial sequence
  siRNA antisense strand oligonucleotide
  misc_feature (4)..(4): n=uracil
  misc_feature (14)..(14): n=uracil
  misc_feature (18)..(18): n=uracil
ccanggccaccggngccnctt

SEQ ID NO: 40 - DNA, 21 bp, Artificial sequence
  siRNA antisense strand oligonucleotide
  misc_feature (3)..(3): n=uracil
  misc_feature (16)..(16): X=uracil
  misc_feature (16)..(16): n=uracil
agnccagcagcagcgngcgtt

SEQ ID NO: 41 - DNA, 21 bp, Artificial sequence
  siRNA antisense strand oligonucleotide
gcggcgccgcagcagcgcgtt

SEQ ID NO: 42 - DNA, 21 bp, Artificial sequence
  siRNA antisense strand oligonucleotide
  misc_feature (2)..(2): n=uracil
  misc_feature (7)..(7): n=uracil
  misc_feature (17)..(17): n=uracil
cnggagncggagcggcnggtt

SEQ ID NO: 43 - DNA, 21 bp, Artificial sequence
  siRNA antisense strand oligonucleotide
  misc_feature (9)..(9): n=uracil
ggggcagcnggcggacacgtt

SEQ ID NO: 44 - DNA, 21 bp, Artificial sequence
  siRNA antisense strand oligonucleotide
  misc_feature (2)..(2): n=uracil
  misc_feature (5)..(5): n=uracil
  misc_feature (15)..(15): n=uracil
  misc_feature (18)..(18): n=uracil
gnccnccacaggganacnctt

SEQ ID NO: 45 - DNA, 21 bp, Artificial sequence
  siRNA antisense strand oligonucleotide
  misc_feature (3)..(3): n=uracil
  misc_feature (6)..(6): n=uracil
  misc_feature (8)..(8): n=uracil
  misc_feature (15)..(15): n=uracil
  misc_feature (17)..(17): n=uracil
acngancnccaccancnggtt

SEQ ID NO: 46 - DNA, 21 bp, Artificial sequence
  siRNA antisense strand oligonucleotide
  misc_feature (10)..(10): n=uracil
  misc_feature (15)..(15): n=uracil
  misc_feature (17)..(18): n=uracil
cacccggccnccgcngnnctt

SEQ ID NO: 47 - DNA, 21 bp, Artificial sequence
  siRNA antisense strand oligonucleotide
  misc_feature (13)..(13): n=uracil
  misc_feature (18)..(18): n=uracil
cagcacccggccnccgcngtt

SEQ ID NO: 48 - DNA, 21 bp, Artificial sequence
  siRNA antisense strand oligonucleotide
  misc_feature (3)..(3): n=uracil
  misc_feature (17)..(17): n=uracil
gangaccccccggcgcngctt

SEQ ID NO: 49 - DNA, 21 bp, Artificial sequence
  siRNA antisense strand oligonucleotide
  misc_feature (2)..(2): n=uracil
  misc_feature (9)..(9): n=uracil
  misc_feature (15)..(15): n=uracil
cngccccangaaacngaagtt

SEQ ID NO: 50 - DNA, 21 bp, Artificial sequence
  siRNA antisense strand oligonucleotide
  misc_feature (11)..(11): n=uracil
  misc_feature (13)..(13): n=uracil
agagcccccangngggggctt

SEQ ID NO: 51 - DNA, 21 bp, Artificial sequence
  siRNA antisense strand oligonucleotide
  misc_feature (12)..(12): n=uracil
aaagaccagcangccggcatt

SEQ ID NO: 52 - DNA, 21 bp, Artificial sequence
  siRNA antisense strand oligonucleotide
  misc_feature (16)..(17): n=uracil
agagcagacacacccnnggtt

SEQ ID NO: 53 - DNA, 21 bp, Artificial sequence
  siRNA antisense strand oligonucleotide
ggaaagagcagacacaccctt

SEQ ID NO: 54 - DNA, 21 bp, Artificial sequence
  siRNA antisense strand oligonucleotide
  misc_feature (3)..(3): n=uracil
  misc_feature (5)..(5): n=uracil
  misc_feature (10)..(10): n=uracil
  misc_feature (12)..(12): n=uracil
  misc_feature (16)..(16): n=uracil
  misc_feature (19)..(19): n=uracil
ggnanagcgncnagcngantt

SEQ ID NO: 55 - DNA, 21 bp, Artificial sequence
  siRNA antisense strand oligonucleotide
  misc_feature (2)..(3): n=uracil
  misc_feature (4)..(4): n=uracil
  misc_feature (10)..(10): n=uracil
cncncaggcngccaagggctt

SEQ ID NO: 56 - DNA, 21 bp, Artificial sequence
  siRNA antisense strand oligonucleotide
  misc_feature (3)..(4): X=uracil
  misc_feature (6)..(6): n=uracil
  misc_feature (13)..(13): n is a, c, g, or t
  misc_feature (14)..(14): n=uracil
  misc_feature (16)..(17): n=uracil
ggnngncacagcnngnncctt

SEQ ID NO: 57 - DNA, 21 bp, Artificial sequence
  siRNA antisense strand oligonucleotide
  misc_feature (1)..(1): n=uracil
  misc_feature (4)..(4): n=uracil
  misc_feature (7)..(8): n=uracil
  misc_feature (10)..(10): n=uracil
  misc_feature (17)..(18): n=uracil
nccnggnngncacagcnngtt

SEQ ID NO: 58 - DNA, 21 bp, Artificial sequence
  siRNA antisense strand oligonucleotide
  misc_feature (4)..(4): n=uracil
  misc_feature (7)..(7): n=uracil
  misc_feature (10)..(11): n=uracil
  misc_feature (13)..(13): n=uracil
ggcnccnggnngncacagctt

SEQ ID NO: 59 - DNA, 21 bp, Artificial sequence
  siRNA antisense strand oligonucleotide
  misc_feature (14)..(14): n=uracil
  misc_feature (17)..(17): n=uracil
ccacagacagggcnccnggtt

SEQ ID NO: 60 - DNA, 21 bp, Artificial sequence
  siRNA antisense strand oligonucleotide
  misc_feature (1)..(2): n=uracil
nnccccaggaacacagggctt

SEQ ID NO: 61 - DNA, 21 bp, Artificial sequence
  siRNA antisense strand oligonucleotide
  misc_feature (4)..(5): n=uracil
  misc_feature (12)..(12): n=uracil
cacnncccaagnccccagctt

SEQ ID NO: 62 - DNA, 21 bp, Artificial sequence
  siRNA antisense strand oligonucleotide
  misc_feature (5)..(5): n=uracil
  misc_feature (16)..(16): n=uracil
aacangacacacccancactt

SEQ ID NO: 63 - DNA, 21 bp, Artificial sequence
  siRNA antisense strand oligonucleotide
  misc_feature (2)..(2): n=uracil
  misc_feature (7)..(7): n=uracil
  misc_feature (17)..(17): n=uracil
gncagcngagcacaaanaatt

SEQ ID NO: 64 - DNA, 21 bp, Artificial sequence
  siRNA antisense strand oligonucleotide
  misc_feature (18)..(18): n=uracil
ggaagcgggggagggagngtt

SEQ ID NO: 65 - DNA, 21 bp, Artificial sequence
  siRNA antisense strand oligonucleotide
  misc_feature (1)..(1): n=uracil
  misc_feature (5)..(5): n=uracil
  misc_feature (7)..(7): n=uracil
  misc_feature (12)..(12): n=uracil
  misc_feature (15)..(15): n=uracil
  misc_feature (19)..(19): n=uracil
naaananaaagngcnaagntt

SEQ ID NO: 66 - DNA, 21 bp, Artificial sequence
  siRNA antisense strand oligonucleotide
  misc_feature (1)..(1): n=uracil
  misc_feature (3)..(4): n=uracil
  misc_feature (9)..(9): n=uracil
  misc_feature (11)..(11): n=uracil
  misc_feature (14)..(14): n=uracil
  misc_feature (18)..(18): n=uracil
nanngacananccngaangtt

SEQ ID NO: 67 - DNA, 53 bp, Artificial sequence
  siRNA oligonucleotide
  misc_feature (6)..(6): n=uracil
  misc_feature (15)..(15): n=uracil
  misc_feature (18)..(18): n=uracil
  misc_feature (20)..(21): n=uracil
  misc_feature (37)..(37): n=uracil
  misc_feature (48)..(53): n=uracil
ggcgcngggcacgcngcngnncaagagacagcagcgngcccagcgccnnnnnn

SEQ ID NO: 68 - DNA, 3 bp, Artificial sequence
  siRNA loop sequence
ccc

SEQ ID NO: 69 - DNA, 4 bp, Artificial sequence
  siRNA loop sequence
  misc_feature (1)..(2): n=uracil
nncg

SEQ ID NO: 70 - DNA, 5 bp, Artificial sequence
  siRNA loop sequence
ccacc

SEQ ID NO: 71 - DNA, 6 bp, Artificial sequence
  siRNA loop sequence
  misc_feature (2)..(2): n=uracil
cncgag

SEQ ID NO: 72 - DNA, 6 bp, Artificial sequence
  siRNA loop sequence
  misc_feature (5)..(6): n=uracil
aagcnn

SEQ ID NO: 73 - DNA, 7 bp, Artificial sequence
  siRNA loop sequence
ccacacc

SEQ ID NO: 74 - DNA, 9 bp, Artificial sequence
  siRNA loop sequence
  misc_feature (1)..(2): n=uracil
nncaagaga

SEQ ID NO: 75 - protein, 9 aa, Artificial sequence
  Pac-1 antigenic epitope
RSDSRAPVY

SEQ ID NO: 76 - protein, 9 aa, Artificial sequence
  Pac-1 antigenic epitope
LRDPREAER

SEQ ID NO: 77 - protein, 9 aa, Artificial sequence
  Pac-1 antigenic epitope
LDEAFDFVK

SEQ ID NO: 78 - protein, 9 aa, Artificial sequence
  Pac-1 antigenic epitope
ELECAALGT

SEQ ID NO: 79 - protein, 9 aa, Artificial sequence
  Pac-1 antigenic epitope
QLLQFETQV

SEQ ID NO: 80 - protein, 9 aa, Artificial sequence
  Pac-1 antigenic epitope
RLDEAFDFV

SEQ ID NO: 81 - protein, 9 aa, Artificial sequence
  Pac-1 antigenic epitope
LLLDCRPFL

SEQ ID NO: 82 - protein, 9 aa, Artificial sequence
  Pac-1 antigenic epitope
LLDCRPFLA

SEQ ID NO: 83 - protein, 9 aa, Artificial sequence
  Pac-1 antigenic epitope
VLDEGSASV

SEQ ID NO: 84 - protein, 9 aa, Artificial sequence
  Pac-1 antigenic epitope
LMQSRRVRL

SEQ ID NO: 85 - protein, 9 aa, Artificial sequence
  Pac-1 antigenic epitope
LLQFETQVL

SEQ ID NO: 86 - DNA, 63 bp, Artificial sequence
  cDNA synthesis primer
ggccagtgaattgtaatacgactcactatagggaggcggtttttttttttttttttttttttt

(19) United States
(12) Patent Application Publication
(10) Pub. No.: US 2007/0054321 A1
(43) Pub. Date: Mar. 8, 2007

(54) Methods of diagnosing and treating inflammatory diseases using pac-1 (dusp2)

(75) Inventors: Kate Jeffrey, Clovelly (AU); Charles Mackay, Vaucluse (AU); Michael Rolph, Kogarah (AU)

Correspondence Address: MARSHALL, GERSTEIN & BORUN LLP, 233 S. WACKER DRIVE, SUITE 6300, SEARS TOWER, CHICAGO, IL, 60606, US

(73) Assignee: G2 THERAPIES LTD., DARLINGTONHURST (AU)

(21) Appl. No.: 10/545,696

(22) PCT Filed: Feb. 12, 2004
(86) PCT No.: PCT/AU04/00168

§ 371 Date: Sep. 8, 2006

(30) Foreign Application Priority Data
Feb. 12, 2003 (AU) 2003900639

(51) Int. Cl. G01N 33/567; C12Q 1/48

(52) U.S. Cl. 435/7.2; 435/15

(57) ABSTRACT

The present invention relates generally to the diagnosis and treatment of inflammatory diseases such as rheumatoid arthritis and asthma in humans and other animals. The invention also relates to a method for identifying agonists and antagonists of the enzyme PAC-1 (DUSP2) that are useful in the therapeutic methods described herein. The present invention relates generally to the diagnosis and treatment of inflammatory diseases such as rheumatoid arthritis and asthma in humans and other animals. The invention also relates to a method for identifying agonists and antagonists of the enzyme PAC-1 (DUSP2) that are useful in the therapeutic methods described herein.

### FIELD OF THE INVENTION

The present invention relates generally to the diagnosis and treatment of inflammatory diseases such as rheumatoid arthritis and asthma in humans and other animals. The invention also relates to a method for identifying agonists and antagonists of the enzyme PAC-1 (DUSP2) that are useful in the therapeutic methods described herein.

### BACKGROUND OF THE INVENTION

Chronic inflammatory diseases such as rheumatoid arthritis (RA) are debilitating diseases affecting millions of people.

RA is a systemic, chronic, inflammatory disease affecting approximately 1-2% of the world's population. The manifestations of RA are usually most severe in the joints. The most frequently affected joints are the proximal interphalangeal and metacarpophalangeal joints in the hands and the metatarsophalangeal joints in the feet. The shoulders, elbows, knees, ankles, and wrists are also common targets of RA. Joints affected by RA (“active” joints) are characteristically tender. Accordingly, RA sufferers often experience pain and impaired mobility. While the severity and progression of RA vary considerably among affected individuals, RA patients as a group have twice the mortality rate of their unaffected counterparts. This decrease in life expectancy appears to be due in part to the side effects of current RA treatments.

In the initial stages of RA, the synovium of the affected joint becomes enlarged and inflamed. Expansion of the synovium is accompanied by angiogenesis and neovascularization. This, in turn, facilitates infiltration into the area by plasma cells, lymphocytes, and macrophages. As inflammatory cells accumulate within it, the synovium becomes edematous and hyperplastic. Neutrophils infiltrate the synovial fluid and cluster on the surface of the synovium. Fibrin deposition also occurs in the joint space. The synovial fluid increases in volume and turbidity with the accumulation of neutrophils, mononuclear cells and occasional red blood cells.

Chronic inflammation causes the synovium to thicken and extend over the articular surface, forming villi which project into the joint space. This aberrant and highly vascularized structure is called a pannus. The pannus invades and erodes the underlying cartilage, with the most active cartilage destruction occurring at the interface between pannus and cartilage. Cartilage destruction is effectuated by collagenase and metallo-proteinases secreted by the inflammatory cells of the pannus. Erosion eventually extends to the subchondral bone, articular capsule and ligaments. Osteoclastic molecules released by the inflammatory cells and synoviocytes allow the synovium to penetrate into the bone and form juxto-articular erosions, subchondral cysts, and osteoporosis.

After the cartilage has been destroyed, the pannus fills the entire joint space, laying down fibrous bands which bridge the opposing bones. As this fibrous ankylosis becomes calcified, a resulting bony ankylosis forms which fuses the opposing bones and prevents the joint from functioning.

RA is an autoimmune disease wherein certain patients have antinuclear antibodies and antibodies directed at autologous proteins such as immunoglobulin G (IgG), collagen, and cytoskeletal filamentous proteins. Rheumatoid factors (RFs) are autoantibodies found in serum and synovial fluid which bind to the Fc portion of IgG. RFs have been implicated in the pathogenesis of RA. When RFs bind IgGs, complement-activating immune complexes are formed. Activated complement causes the release of vasoactive and chemotactic substances which attract neutrophils and macrophages to the immune complex. As these cells ingest and destroy the immune complexes, they release molecules which cause inflammation and protein degradation and which attract other cells that contribute to RA pathogenesis. Because RFs are synthesized in joints and RFs of the IgG class are self-associating, immune complexes are frequently localized to joints. Nevertheless, immune complexes do circulate in some patients and are thought to cause the extra-articular manifestations of RA such as rheumatoid nodules, reactive amyloidosis and acute vasculitis.

The evidence that RA has autoimmune origins is supported by the finding that injecting avian type II collagen into rats or mice causes them to develop antibodies that recognize the rodent's endogenous collagen. The resulting auto-immune response, which is known as “collagen arthritis,” mimics the physiological symptoms of RA such as joint inflammation (Trentham et al., J. Exp. Med., 146: 857 (1977); Courtenay et al., Nature, 283: 666 (1980)). Collagen arthritis is a well established model system for the study of RA (Staines & Wooley, Br. J. Rheumatology, 1994, 33: 798 (1994)).

The primary method for treating RA is through the administration of drugs which suppress the immune system or reduce inflammation. The two main classes of inflammation inhibitors are corticosteroids and non-steroidal anti-inflammatory drugs (NSAIDs).

Corticosteroids are not favored RA medications because, although they can achieve dramatic short-term improvements in RA, long-term treatment is not advisable due to serious side effects and diminished effectiveness of the drug. Short-term corticosteroid treatment is also far from ideal because arthritis symptoms rapidly reappear, often with increased severity, after treatment has stopped. Although corticosteroids such as prednisone can suppress clinical symptoms of RA, the drugs do not prevent RA-mediated joint destruction. Furthermore, typical corticosteroid side effects include: peptic ulcer, hypertension, diabetes mellitus, and glaucoma.

NSAIDs such as aspirin, ibuprofen, and indomethacin are frequently prescribed for RA. Although these drugs can reduce swelling in active joints by inhibiting prostaglandin synthesis, their poor penetration of joint spaces necessitates administration of high doses, and such high doses tend to cause gastrointestinal irritation, ulceration, and bleeding. NSAIDs affect prostaglandin-regulate processes not associated with the inflammation process. NSAIDs are also potent renal toxins, and so are also inadequate for treatment of RA.

Patients with severe active RA may be treated with immunosuppressants such as cyclophosphamide, methotrexate, and azathioprine. While they can reduce inflammation, these drugs affect the patient's entire immune system and have serious side effects including liver disease, bone marrow suppression, and increased risk of malignancy.

Other RA medications include penicillamine, chloroquine, hydroxychloroquine, and gold salts. All have potentially serious side effects. D-penicillamine can cause bone marrow suppression, proteinuria, and nephrosis. Deaths resulting from penicillamine treatment have also been reported. Patients treated with hydroxychloroquine or chloroquine must be monitored for signs of irreversible retinal damage. Gold can induce toxic reactions in the form of dermatitis, renal failure or hepatitis, and its efficacy at treating arthritis is questionable.

A treatment method for chronic inflammatory diseases such as RA is therefore highly desirable. There is a need for a compound which inhibits the signs and symptoms of these diseases, and particularly reduces the pain, inflammation, joint swelling, and lesions associated with these diseases.

Phosphatase of Activated Cells (PAC-1) is a MAP kinase phosphatase. In particular, PAC-1 is a member of a family of dual specific phosphatases (DUSPs) and is otherwise known as dual specific phosphatase 2 (DUSP2). DUSPs are thought to inactivate target kinases by dephosphorylating both the phosphothreonine and phosphotyrosine residues.

There are currently eleven identified members of the DUSP family, all differing in their tissue distribution, subcellular location, substrate specificities and transcriptional control (Camps et al., FASEB J., 14: 6(2000); Theodosiou & Ashworth, Genome Biol., 3: 7 (2002)). The DUSPs fall into three main categories: those that consist of 4 exons, are induced by growth or stress and are located in the nucleus (DUSPs 1-5), those that consist of 3 exons, are located in the cytosol and are not directly controlled by transcription (DUSP 6, 7, 9, 10) and those that consist of 6 exons (DUSP 8 and 16). DUSPs 1-5 have about 80% homology with a conserved carboxyl terminus containing the catalytic domain and a more variable amino terminus that is involved in substrate recognition (Camps et al., 2000, supra).

PAC-1 is thought to negatively regulate members of the mitogen-activated protein (MAP) kinase superfamily (MAPK/ERK, SAPK/JNK, p38), which are associated with cellular proliferation and differentiation.

### SUMMARY OF THE INVENTION

In work leading up to the present invention, the inventors studied the role of the enzyme PAC-1 in inflammatory diseases such as rheumatoid arthritis. The inventors have shown that PAC-1 expression is substantially increased in human mast cells activated with IgE. This finding is significant because mast cells have been implicated as a cellular link between autoantibodies, soluble mediators and other effector populations in inflammatory arthritis.

Accordingly, in a first aspect the present invention provides a method of screening for a compound that suppresses or reduces inflammation, the method comprising determining the activity of PAC-1 in the presence and absence of a candidate compound, wherein altered PAC-1 activity in the presence of the compound indicates that the compound suppresses or reduces inflammation.

In a preferred embodiment of this method of screening, reduced PAC-1 activity in the presence of the compound indicates that the compound suppresses or reduces inflammation.

In a further embodiment of this method of screening, PAC-1 activity is determined by measuring the phosphatase activity of PAC-1.

In another aspect, the present invention provides a method of screening for a compound that suppresses or reduces inflammation, the method comprising determining the expression levels of PAC-1 in the presence and absence of a candidate compound, wherein altered PAC-1 expression in the presence of the compound indicates that the compound suppresses or reduces inflammation.

In a preferred embodiment of this method of screening, reduced PAC-1 expression in the presence of the compound indicates that the compound suppresses or reduces inflammation.

In a further embodiment of this aspect, the method involves exposing a translation system capable of expressing PAC-1 to a candidate compound and comparing the levels of expression of PAC-1 in the presence of the compound to the levels achieved under similar conditions but in the absence of the compound. The translation system may be a cell-free translation system. Alternatively, the translation system may comprise eukaryotic or prokaryotic cells.

In a further aspect the present invention provides a method of screening for a compound that suppresses or reduces inflammation, the method comprising determining the ability of a candidate compound to modulate the binding of PAC-1 to a PAC-1 substrate, wherein an altered level of binding of PAC-1 to the substrate in the presence of the compound indicates that the compound suppresses or reduces inflammation.

In a preferred embodiment of this method of screening, a reduced level of binding of PAC-1 to the substrate in the presence of the compound indicates that the compound suppresses or reduces inflammation.

In one embodiment of this aspect, the PAC-1 substrate is selected from the group consisting of ERK, p38 and JNK.

In a further aspect the present invention provides a method of screening for a compound that promotes an immune response, the method comprising determining the activity of PAC-1 in the presence and absence of a candidate compound, wherein altered PAC-1 activity in the presence of the compound indicates that the compound promotes an immune response.

In a preferred embodiment of this aspect, enhanced or increased PAC-1 activity in the presence of the compound indicates that the compound promotes an immune response.

In another aspect, the present invention provides a method of screening for a compound that promotes an immune response, the method comprising determining the expression levels of PAC-1 in the presence and absence of a candidate compound, wherein altered PAC-1 expression in the presence of the compound indicates that the compound promotes an immune response.

In a preferred embodiment of this aspect, enhanced or increased PAC-1 expression in the presence of the compound indicates that the compound promotes an immune response.

In a further aspect the present invention provides a method of screening for a compound that promotes an immune response, the method comprising determining the ability of a candidate compound to modulate the binding of PAC-1 to a PAC-1 substrate, wherein an altered level of binding of PAC-1 to the substrate in the presence of the compound indicates that the compound promotes an immune response.

In a preferred embodiment of this aspect, an enhanced or increased level of binding of PAC-1 to the substrate indicates that the compound promotes an immune response.

In a preferred embodiment of the present invention, the candidate compound is selected from the group consisting of a peptide, such as a peptide derived from PAC-1, a PAC-1 dominant-negative mutant, an antibody directed against PAC-1, non-peptide inhibitors of PAC-1 such as small organic molecules, antisense compounds directed against PAC-1-encoding mRNA, anti-PAC-1 catalytic molecules such as ribozymes or a DNAzymes, dsRNA or small interfering RNA (RNAi) molecules that target PAC-1 expression.

In one embodiment of the present invention, the candidate compound is obtained from expression products of a gene library, a low molecular weight compound library (such as the low molecular weight compound library of ChemBridge Research Laboratories), a cell extract, microorganism culture supernatant, bacterial cell components and the like.

In yet another aspect, the present invention provides a method for treating or preventing an inflammatory disease in a subject, the method comprising administering to the subject a compound that modulates PAC-1 activity in an amount effective to inhibit or reduce inflammation.

In a preferred embodiment of this aspect the method comprises administering to the subject a compound that reduces or inhibits PAC-1 activity in an amount effective to inhibit or reduce inflammation. Preferably, the compound specifically reduces or inhibits PAC-1 activity.

By “specifically reduces or inhibits PAC-1 activity” we mean that the compound significantly reduces or inhibits PAC-1 activity without significantly reducing or inhibiting the activity of other DUSPs.

In yet another aspect, the present invention provides a method for treating or preventing an inflammatory disease in a subject, the method comprising administering to the subject a compound that alters the level of expression of functional PAC-1 in an amount effective to inhibit or reduce inflammation.

In a preferred embodiment of this aspect the method comprises administering to the subject a compound that reduces or inhibits functional PAC-1 expression in an amount effective to inhibit or reduce inflammation. Preferably, the compound specifically reduces or inhibits PAC-1 expression.

By “specifically reduces or inhibits PAC-1 expression” we mean that the compound significantly reduces or inhibits PAC-1 expression without significantly reducing or inhibiting the expression of other DUSPs.

In yet another aspect, the present invention provides a method for treating or preventing immunosuppression in a subject, the method comprising administering to the subject a compound that alters the activity of PAC-1 in an amount effective to treat immunosuppression.

In a preferred embodiment of this aspect the method comprises administering to the subject a compound that increases or enhances PAC-1 activity in an amount effective to treat immunosuppression. Preferably, the compound specifically increases or enhances PAC-1 activity.

By “specifically increases or enhances PAC-1 activity” we mean that the compound significantly increases or enhances PAC-1 activity without significantly increasing or enhancing the activity of other DUSPs.

In yet another aspect, the present invention provides a method for treating or preventing immunosuppression in a subject, the method comprising administering to the subject a compound that alters the levels of expression of PAC-1 in an amount effective to treat immunosuppression.

In a preferred embodiment of this aspect the method comprises administering to the subject a compound that increases or enhances PAC-1 expression in an amount effective to treat immunosuppression. Preferably, the compound specifically increases or enhances PAC-1 expression.

By “specifically increases or enhances PAC-1 expression” we mean that the compound significantly increases or enhances PAC-1 activity without significantly increasing or enhancing the expression of other DUSPs.

As will be readily understood by those skilled in this field the methods of the present invention provide a rational method for designing and selecting compounds including antibodies which interact with and modulate the activity of PAC-1. In the majority of cases these compounds will require further development in order to increase activity. It is intended that in particular embodiments the methods of the present invention includes such further developmental steps. For example, it is intended that embodiments of the present invention further include manufacturing steps such as incorporating the compound into a pharmaceutical composition in the manufacture of a medicament.

Accordingly, in a further aspect, the method further comprises formulating the identified compound for administration to a human or a non-human animal as described herein.

The present invention also relates to methods for detecting the expression of PAC-1 polynucleotides or polypeptides in a sample (e.g., tissue or sample). Such methods can, for example, be utilized as part of prognostic and diagnostic evaluation of inflammatory disorders and for the identification of subjects exhibiting a predisposition to such disorders.

As used herein, the term “diagnosis”, and variants thereof, such as, but not limited to “diagnose”, “diagnosed” or “diagnosing” shall not be limited to a primary diagnosis of a clinical state, however should be taken to include any primary diagnosis or prognosis of a clinical state. For example, the “diagnostic assay” formats described herein are equally relevant to monitoring inflammatory disease recurrence, or monitoring the efficiency of treatment of the disease. All such uses of the assays described herein are encompassed by the present invention.

In a preferred embodiment, the present invention provides a method of diagnosing disorders associated with PAC-1-expressing cells comprising the step of measuring the expression patterns of PAC-1 protein and/or mRNA. Yet another embodiment of a method of diagnosing disorders associated with PAC-1-expressing cells comprising the step of detecting PAC-1 expression using anti-PAC-1 antibodies. Such methods of diagnosis encompass the use of compositions, kits and other approaches for determining whether a patient is a candidate for treatment in which PAC-1 is targeted.

In one particular embodiment the present invention provides a method of detecting a PAC-1-associated transcript in a biological sample, the method comprising contacting the biological sample with a polynucleotide that selectively hybridizes to a sequence at least 80% identical to a sequence as shown in SEQ ID NO:1 or 3. Preferably the percentage identity to a sequence disclosed in SEQ ID NO:1 or 3 is at least about 85% or 90% or 95%, and still more preferably at least about 98% or 99%.

In a further preferred embodiment, the present invention provides a method of diagnosing an inflammatory disorder in a human or animal subject being tested, the method comprising contacting a biological sample from the subject being tested with a nucleic acid probe for a time and under conditions sufficient for hybridization to occur and then detecting the hybridization wherein a modified level of hybridization of the probe for the subject being tested compared to the hybridization obtained for a control subject not having an inflammatory disorder indicates that the subject being tested has an inflammatory disorder, and wherein the nucleic acid probe comprises a sequence selected from the group consisting of:
(i) a sequence comprising at least about 20 contiguous nucleotides from SEQ ID NO:1 or 3;
(ii) a sequence that hybridizes under at least low stringency hybridization conditions to at least about 20 contiguous nucleotides from SEQ ID NO:1 or 3;
(iii) a sequence that is at least about 80% identical to SEQ ID NO:1 or 3;
(iv) a sequence that encodes an amino acid sequence as shown in SEQ ID NO:2 or 4; and
(v) a sequence that is complementary to any one of the sequences set forth in (i) or (ii) or (iii) or (iv).

As used herein, the term “modified level” includes an enhanced, increased or elevated level of an integer being assayed, or alternatively, a reduced or decreased level of an integer being assayed.

In a preferred embodiment of the invention, an elevated, enhanced or increased level of expression of the nucleic acid is indicative of an inflammatory disorder.

Both classical hybridization and amplification formats, and combinations thereof, are encompassed by the invention. In one embodiment, the hybridization comprises performing a nucleic acid hybridization reaction between a labeled probe and a second nucleic acid in the biological sample from the subject being tested, and detecting the label. In another embodiment, the hybridization comprising performing a nucleic acid amplification reaction eg., polymerase chain reaction (PCR), wherein the probe consists of a nucleic acid primer and nucleic acid copies of the nucleic acid in the biological sample are amplified. As will be known to the skilled artisan, amplification may proceed classical nucleic acid hybridization detection systems, to enhance specificity of detection, particularly in the case of less abundant mRNA species in the sample.

In a preferred embodiment, the polynucleotide is immobilised on a solid surface.

In a related embodiment, the present invention provides a method of detecting a PAC-1 polypeptide in a biological sample the method comprising contacting the biological sample with an antibody that binds specifically to PAC-1.

In a preferred embodiment, the present invention provides a method of diagnosing an inflammatory disorder in a human or animal subject being tested, the method comprising contacting a biological sample from the subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having an inflammatory disorder indicates that the subject being tested has an inflammatory disorder, and wherein the antibody binds specifically to a polypeptide having an amino acid sequence comprising at least about 10 contiguous amino acid residues of a sequence having at least about 80% identity to a sequence as shown in SEQ ID NO:2 or SEQ ID NO:4.

In one embodiment an elevated, enhanced or increased level of the antigen-antibody complex is indicative of an inflammatory disorder.

The present invention is not to be limited by the source or nature of the biological sample. In one embodiment, the biological sample is from a patient undergoing a therapeutic regimen to treat an an inflammatory disorder. In an alternative preferred embodiment, the biological sample is from a patient suspected of having or developing and inflammatory disorder.

In a preferred embodiment, the biological tissue sample comprises mast cells, T cells or eosinophils. In a further embodiment, the tissue sample may be synovial fluid, blood or lung sputum.

In another aspect the present invention provides a method of monitoring the efficacy of a therapeutic treatment of an inflammatory disorder, the method comprising:
(i) providing a biological sample from a patient undergoing the therapeutic treatment; and
(ii) determining the level of a PAC-1-associated transcript in the biological sample by contacting the biological sample with a polynucleotide that selectively hybridizes to a sequence having at least about 80% identity to a sequence as shown in SEQ ID NO:1 or SEQ ID NO:3, thereby monitoring the efficacy of the therapy.

Preferably the method further comprises comparing the level of the PAC-1-associated transcript to a level of the PAC-1-associated transcript in a biological sample from the patient prior to, or earlier in, the therapeutic treatment.

In a related embodiment, the present invention provides a method of monitoring the efficacy of a therapeutic treatment of an inflammatory disorder, the method comprising:
(i) providing a biological sample from a patient undergoing the therapeutic treatment; and
(ii) determining the level of a PAC-1 polypeptide in the biological sample by contacting the biological sample with an antibody, wherein the antibody specifically binds to a polypeptide sequence as shown in SEQ ID NO:2 or SED ID NO:4, thereby monitoring the efficacy of the therapy.

Preferably the method further comprises comparing the level of the PAC-1 polypeptide to a level of the PAC-1 polypeptide in a biological sample from the patient prior to, or earlier in, the therapeutic treatment.

The present invention also provides kits comprising polynucleotide probes and/or monoclonal antibodies, and optionally quantitative standards, for carrying out methods of the invention. Furthermore, the invention provides methods for evaluating the efficacy of drugs, and monitoring the progress of patients, involved in clinical trials for the treatment of disorders as recited herein.

As will be apparent, preferred features and characteristics of one aspect of the invention are applicable to other aspects of the invention.

### BRIEF DESCRIPTION OF THE FIGURES

FIG. 1 shows Dual Specificity Phosphatase (DUSP1, DUSP2 (PAC-1), DUSP4 and DUSP5) expression in human cord blood-derived mast cells activated with IgE for 0-6 hrs (n=1).

FIG. 2 shows results of histological analysis of ankle tissue from (A) wild type (WT) and (3) PAC-1 deficient (PAC-1 −/−) mice injected with the arthritic serum (K/B×N serum). Histological analysis of ankle tissue from wild type (WT) mice injected with wild type serum (C) is included as a control.

FIG. 3 shows results of a Western blot analysis of PAC-1 substrate in protein samples form activated macrophages derived from wild type (WT) and PAC-1 deficient (PAC-1 −/−) mice. (A) Detection of levels of p38 using an anti-p38 antibody. (B) Detection of levels of ERK using an anti-ERK antibody.

FIG. 4 shows the production of inflammatory mediators NO (A) PGE2 (B), NOS-2 (C) and COX-2 (D) in stimulated macrophages of wild type (WT) and PAC-1 deficient (PAC-1 −/−) mice.

FIG. 5 shows the production of inflammatory mediators TNF-α (A) and IL-6 (B) in stimulated macrophages of wild type (WT) and PAC-1 deficient (PAC-1 −/−) mice.

### KEY TO THE SEQUENCE LISTING

SEQ ID NO:1—cDNA encoding human PAC-1.
SEQ ID NO:2—Human PAC-1.
SEQ ID NO:3—cDNA encoding murine PAC-1.
SEQ ID NO:4—Murine PAC-1.
SEQ ID NO's 5 to 66—Polynucleotides for producing siRNA molecules which downregulated human PAC-1 production.
SEQ ID NO:67—Pac-1 siRNA
SEQ ID NO's 68 to 74—Stem loop sequences of polynucleotides for producing siRNA molecules which downregulated human PAC-1 production.
SEQ ID NO's 75 to 85—Antigenic epitopes of human PAC-1.
SEQ ID NO:86—cDNA synthesis primer.

### DETAILED DESCRIPTION OF THE PREFERRED EMBODIMENTS

### General Techniques

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Inmunology, John Wiley & Sons (including all updates until present), and are incorporated herein by reference.

PAC-1

As used herein, the term “PAC-1” refers to any peptide, polypeptide, or protein having at least about 80% amino acid sequence identity to the amino acid sequence of a human or mouse PAC-1 polypeptide set forth in SEQ ID NO:2 or SEQ ID NO:4. Preferably, the percentage identity to SEQ ID NO:2 or 4 is at least about 85%, more preferably at least about 90%, even more preferably at least about 95% and still more preferably at least about 99%. The term “PAC-1” shall also be taken to include a peptide, polypeptide or protein fragment having the known biological activity of full length PAC-1, or the known binding specificity of full length PAC-1.

PAC-1 Antagonists/Agonists

The range of compounds contemplated herein include PAC-1 agonists or antagonists of a biological function of PAC-1. Examples of suitable types of these compounds are described below.

Protein or Peptide Inhibitors

In another embodiment, the candidate compounds are proteins. By “protein” in this context it is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or (L)-configuration. If non-naturally occurring side chains are used, non-amino acid substituents may be used, for example to prevent or retard in vivo degradations.

In a further preferred embodiment, the candidate compounds are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made. Particularly preferred in this embodiment are libraries of bacterial, fungal, viral, and mammalian proteins, with the latter being preferred, and human proteins being especially preferred.

In a further preferred embodiment, the candidate compounds are peptides of from about 5 to about 30 amino acids, with from about 5 to about 20 amino acids being preferred, and from about 7 to about 15 being particularly preferred. The peptides may be digests of naturally occurring proteins, random peptides, or “biased” random peptides. By “randomized” or grammatical equivalents herein is meant that each peptide consists of essentially random amino acids. Since generally these random peptides are chemically synthesized, they may incorporate any amino acid at any position. The synthetic process can be designed to generate randomized proteins to allow the formation of all or most of the possible combinations over the length of the sequence, thus forming a library of randomized candidate bioactive proteinaceous compounds.

Preparation and screening of combinatorial chemical libraries are well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 69096913), vinylogous polypeptides (Hagihara et al. (1992) J. Amer. Chem. Soc. 114: 6568), nonpeptidal peptidomimetics with a Beta D Glucose scaffolding (Hirschmann et al., (1992) J. Amer. Chem. Soc. 114: 92179218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

In one embodiment, peptidyl PAC-1 inhibitors are chemically or recombinantly synthesized as oligopeptides (approximately 10-25 amino acids in length) derived from the PAC-1 sequence (SEQ ID NO:2 or 4). Alternatively, PAC-1 fragments are produced by digestion of native or recombinantly produced PAC-1 by, for example, using a protease, e.g., trypsin, thermolysin, chymotrypsin, or pepsin. Computer analysis (using commercially available software, e.g. MacVector, Omega, PCGene, Molecular Simulation, Inc.) is used to identify proteolytic cleavage sites. The proteolytic or synthetic fragments can comprise as many amino acid residues as are necessary to partially or completely inhibit PAC-1 function. Preferred fragments will comprise at least 5, 10, 15, 20, 25, 30, 35, 40, 45, 50, 55, 60, 65, 70, 75, 80, 85, 90, 95, 100 or more amino acids in length.

Protein or peptide inhibitors may also be dominant-negative mutants of PAC-1. The term “dominant-negative mutant” refers to a PAC-1 polypeptide that has been mutated from its natural state and that interacts with a protein that PAC-1 normally interacts with thereby preventing endogenous native PAC-1 from forming the interaction.

Anti-PAC-1 Antibodies

The term “antibody” as used in this invention includes intact molecules as well as fragments thereof, such as Fab, F(ab′)2, and Fv which are capable of binding an epitopic determinant of PAC-1. These antibody fragments retain some ability to selectively bind with its antigen and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

Antibodies of the present invention can be prepared using intact PAC-1 or fragments thereof as the immunizing antigen. A peptide used to immunize an animal can be derived from translated cDNA or chemical synthesis and is purified and conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide may then be used to immunize the animal (e.g., a mouse or a rabbit).

If desired, polyclonal antibodies can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1991, incorporated by reference).

Monoclonal antibodies may be prepared using any technique which provides for the production of antibody molecules by continuous cell lines in culture, such as, for example, the hybridoma technique, the human B-cell hybridoma technique, and the EBV-hybridoma technique (Kohler et al. Nature 256, 495-497, 1975; Kozbor et al., J. Immunol. Methods 81, 31-42, 1985; Cote et al., Proc. Natl. Acad. Sci. USA 80, 2026-2030, 1983; Cole et al., Mol. Cell Biol. 62, 109-120, 1984).

Methods known in the art allow antibodies exhibiting binding for PAC-1 to be identified and isolated from antibody expression libraries. For example, a method for the identification and isolation of an antibody binding domain which exhibits binding to PAC-1 is the bacterio-phage a vector system. This vector system has been used to express a combinatorial library of Fab fragments from the mouse antibody repertoire in Escherichia coli (Huse, et al., Science, 246:1275-1281, 1989) and from the human antibody repertoire (Mullinax, et al., Proc. Nat. Acad. Sci., 87:8095-8099, 1990). This methodology can also be applied to hybridoma cell lines expressing monoclonal antibodies with binding for a preselected ligand. Hybridomas which secrete a desired monoclonal antibody can be produced in various ways using techniques well understood by those having ordinary skill in the art and will not be repeated here. Details of these techniques are described in such references as Monoclonal Antibodies-Hybridomas: A New Dimension in Biological Analysis, Edited by Roger H. Kennett, et al., Plenum Press, 1980; and U.S. Pat. No. 4,172,124, incorporated by reference.

In addition, methods of producing chimeric antibody molecules with various combinations of “humanized” antibodies are known in the art and include combining murine variable regions with human constant regions (Cabily, et al. Proc. Natl. Acad. Sci. USA, 81:3273, 1984), or by grafting the murine-antibody complementarity determining regions (CDRs) onto the human framework (Riechmann, et al., Nature 332:323, 1988).

In one embodiment, the antibody binds at least a portion of a region of human PAC-1 selected from, but not limited to, the group consisting of SEQ ID NO's 75-85.

Antisense Compounds

The term “antisense compounds” encompasses DNA or RNA molecules that are complementary to at least a portion of a PAC-1 mRNA molecule (Izant and Weintraub, Cell 36:1007-15, 1984; Izant and Weintraub, Science 229(4711):345-52, 1985) and capable of interfering with a post-transcriptional event such as mRNA translation. Antisense oligomers complementary to at least about 15 contiguous nucleotides of PAC-1-encoding mRNA are preferred, since they are easily synthesized and are less likely to cause problems than larger molecules when introduced into the target PAC-1-producing cell. The use of antisense methods is well known in the art (Marcus-Sakura, Anal. Biochem. 172: 289, 1988). Preferred antisense nucleic acid will comprise a nucleotide sequence that is complementary to at least 15 contiguous nucleotides of a sequence encoding the amino acid sequence set forth in SEQ ID NO:2 or SEQ ID NO:4.

Catalytic Nucleic Acids

The term catalytic nucleic acid refers to a DNA molecule or DNA-containing molecule (also known in the art as a “DNAzyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity (also referred to herein as the “catalytic domain”). To achieve specificity, preferred ribozymes and DNAzymes will comprise a nucleotide sequence that is complementary to at least about 12-15 contiguous nucleotides of a sequence encoding the amino acid sequence set forth in SEQ ID NO: 2 or SEQ ID NO:4.

The types of ribozymes that are particularly useful in this invention are the hammerhead ribozyme (Haseloff and Gerlach, Nature, 334: 585 (1988), Perriman et al., Gene, 133: 157 (1992)) and the hairpin ribozyme (Shippy et al., Mol. Biotech. 12: 117 (1999)).

The ribozymes of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for the ribozymes of this invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

RNA Inhibitors

dsRNA is particularly useful for specifically inhibiting the production of a particular protein. Although not wishing to be limited by theory, Dougherty and Parks (Curr. Opin. Cell Biol. 7: 399 (1995)) have provided a model for the mechanism by which dsRNA can be used to reduce protein production. This model has recently been modified and expanded by Waterhouse et al. (Proc. Natl. Acad. Sci. 95: 13959 (1998)). This technology relies on the presence of dsRNA molecules that contain a sequence that is essentially identical to the mRNA of the gene of interest, in this case an mRNA encoding a PAC-1 protein. Conveniently, the dsRNA can be produced in a single open reading frame in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules targeted agasint PAC-1 is well within the capacity of a person skilled in the art, particularly considering Dougherty and Parks (1995, supra), Waterhouse et al. (1998, supra), WO 99/32619, WO 99/53050, WO 99/49029, and WO 01/34815.

As used herein, the terms “small interfering RNA” (siRNA), and “RNAi” refer to homologous double stranded RNA (dsRNA) that specifically targets a gene product, thereby resulting in a null or hypomorphic phenotype. Specifically, the dsRNA comprises two short nucleotide sequences derived from the target RNA encoding PAC-1 and having self-complementarity such that they can anneal, and interfere with expression of a target gene, presumably at the post-transcriptional level. RNAi molecules are described by Fire et al., Nature 391, 806-811, 1998, and reviewed by Sharp, Genes & Development, 13, 139-141, 1999).

Preferred siRNA molecules comprise a nucleotide sequence that is identical to about 19-21 contiguous nucleotides of the target mRNA. Preferably, the target sequence in PAC-1 mRNA commences with the dinucleotide AA, comprises a GC-content of about 30-70% (preferably, 30-60%, more preferably 40-60% and more preferably about 45%-55%), and does not have a high percentage identity to any nucleotide sequence other than PAC-1 in the genome of the animal in which it is to be introduced, e.g., as determined by standard BLAST search.

The siRNA is preferably capable of downregulating expression of human PAC-1 in a cell. In view of the high percentage conservation between murine and human PAC-1 encoding genes, especially in the coding regions, this should not be taken to indicate a requirement for the siRNA to be specific for human PAC-1-encoding genes. In the cell-based and animal models described herein, it is possible and appropriate in certain circumstances for the siRNA molecules to reduce expression of both endogenous murine PAC-1, as well as ectopically expressed human PAC-1 in the cell. Confirmation of a specific activity of any antagonist against human PAC-1 is determined by assessing the activity of an inhibitor in a cell derived from a PAC-1^{-/-} mouse that has been engineered to express human PAC-1.

As exemplified herein, preferred siRNA against a PAC-1 encoding region comprises a 21-nucleotide sequence set forth in any one of SEQ ID Nos: 5-66. In this respect, SEQ ID Nos: 5-35 each comprise (i) a 19-nucleotide sequence corresponding to a human PAC-1 mRNA target sequence adjacent and downstream of a dinucleotide AA in said mRNA target; and (ii) a 3+-extension dinucleotide TT. SEQ ID NOS: 36-66 each comprise (i) a 19-nucleotide sequence complementary to a human PAC-1 mRNA target sequence contained within SEQ ID NOS: 5-35, respectively; and (ii) a 3′-extension dinucleotide TI.

For producing siRNA which include a stem loop structure from the exemplified siRNAs set forth in SEQ ID NOS: 5-66, the sense and antisense strands are positioned such that they flank an intervening loop sequence. Preferred loop sequences are selected from the group consisting of:

| | | |
|---|---|---|
| (i) | CCC; | (SEQ ID NO: 68) |
| (ii) | UUCG; | (SEQ ID NO: 69) |
| (iii) | CCACC; | (SEQ ID NO: 70) |
| (iv) | CUCGAG; | (SEQ ID NO: 71) |
| (v) | AAGCUU; | (SEQ ID NO: 72) |
| (vi) | CCACACC; | (SEQ ID NO: 73) |
| and | | |
| (vii) | UUCAAGAGA. | (SEQ ID NO: 74) |

Of these loop sequences, the sequence set forth in SEQ ID NO: 74 is particularly preferred for modulating human PAC-1 expression in a cell and/or tissue.

A further example of a siRNA which can be used to down-regulate PAC-1 production in human cells was described by Yin et al. (Nature, 422: 527 (2003) see supplementary information section) and is provided as SEQ ID NO: 67.

Small Molecule Inhibitors

Numerous organic molecules may be assayed for their ability to modulate the immune system. For example, within one embodiment of the invention suitable organic molecules may be selected either from a chemical library, wherein chemicals are assayed individually, or from combinatorial chemical libraries where multiple compounds are assayed at once, then deconvoluted to determine and isolate the most active compounds.

Representative examples of such combinatorial chemical libraries include those described by Agrafiotis et al., “System and method of automatically generating chemical compounds with desired properties,” U.S. Pat. No. 5,463,564; Armstrong, R. W., “Synthesis of combinatorial arrays of organic compounds through the use of multiple component combinatorial array syntheses,” WO 95/02566; Baldwin, J. J. et al., “Sulfonamide derivatives and their use,” WO 95/24186; Baldwin, J. J. et al., “Combinatorial dihydrobenzopyran library,” WO 95/30642; Brenner, S., “New kit for preparing combinatorial libraries.” WO 95/16918; Chenera, B. et al., “Preparation of library of resin-bound aromatic carbocyclic compounds,” WO 95/16712; Ellman, J. A., “Solid phase and combinatorial synthesis of benzodiazepine compounds on a solid support,” U.S. Pat. No. 5,288,514; Felder, E. et al., “Novel combinatorial compound libraries,” WO 95/16209: Lerner. R. et al., “Encoded combinatorial chemical libraries.” WO 93/20242; Pavia, M. R. et al., “A method for preparing and selecting pharmaceutically useful non-peptide compounds from a structurally diverse universal library,” WO 95/04277; Summerton, J. E. and D. D. Weller, “Morpholino-subunit combinatorial library and method,” U.S. Pat. No. 5,506,337; Holmes, C., “Methods for the Solid Phase Synthesis of Thiazolidinones, Metathiazanones, and Derivatives thereof,” WO 96/00148; Phillips, G. B. and G. P. Wei, “Solid-phase Synthesis of Benzimidazoles,” Tet. Letters 37:4887-90, 1996; Ruhland, B. et al., “Solid-supported Combinatorial Synthesis of Structurally Diverse .beta.-Lactams,” J. Amer. Chem. Soc. 111:253-4, 1996; Look, G. C. et al., “The Indentification of Cyclooxygenase-1 Inhibitors from 4-Thiazolidinone Combinatorial Libraries,” Bioorg and Med. Chem. Letters 6:707-12, 1996.

Small molecule inhibitors that allow the reversible and graded regulation of dual specific phosphatases (DUSPS) have recently become available. A number of these inhibitors are based on natural products and may form the basis for new therapeutic agents. Examples of inhibitors of DUSPS are described in Lyon et al, 2002, Nature Reviews, Vol 1:961-976, the entire contents of which are specifically incorporated herein by reference. In the context of the present invention, it is preferred that the inhibitors are specific for DUSP2 (PAC-1).

Methods of Screening for PAC-1 Agonists or Antagonists

Production of Recombinant PAC-1

To generate protein for use in assays for identifying inhibitors or antagonists of PAC-1, the PAC-1 gene may be cloned and expressed in a suitable expression system (such as an E. coli based expression system) and expressed protein may be recovered using an affinity purification strategy. Yields of 10's-100's mg/litre can be expected from this procedure.

The sequence of the PAC-1 gene is known (se SEQ ID NO:s 1 and 3) and primers can be designed to amplify this gene using PCR from mRNA isolated from mast cells or activated T cells. With appropriate restriction sites incorporated into the PCR primers the amplified gene may be cloned directly into an E. coli expression plasmid in-frame with a purification affinity tag sequence (e.g. 6×His, GST, CBD) and downstream of a strong promoter (e.g. T7, T7/lac, tac, trc, PL). After sequencing the gene to ensure that mutations were not introduced during the amplification and cloning steps the recombinant expression plasmid may be transformed into the appropriate host cell for expression.

Host cells may be grown to the desired density and volume before expression is induced. Cells can be harvested when maximum production is reached, lysed by mechanical or chemical means and the contents solubilised if required and subject to affinity purification. The expressed protein may eluted from the affinity resin (e.g. NTA agarose, GST agarose, cellulose). After a single affinity purification step it is expected that the protein will be ˜90% pure. Further purification steps can be performed if necessary.

Screening Protocol Based on MAP-kinase Phosphatase Activity

A number of assays for MAP-kinase phosphatase activity, including PAC-1 activity, have been described and will be known to those skilled in the art. A preferred assay in terms of signal to noise ratio employs the phosphorylation of a substrate called DiFMUP (Molecular Probes). This is a non-specific substrate that can be de-phosphorylated by all Serine/Threonine phosphatases. DiFMUP emits fluorescence after losing a phosphate. One advantage of using the substrate DiFMUP is that other phosphatases, which are commercially available, can also be used to study the specificity of PAC-1 inhibitors.

Fluorescence is measured in a 96/384 well fluorescence plate reader using excitation at 355 nm and emission at 460 nm.

The Molecular Probes assay system is currently available in a 96 well format. This assay sysytem may be improved by, for example, determining the optimal amounts of substrate (DiFMUP), the optimal amounts of phosphatase, and the most suitable buffer conditions in order to develop a good signal to noise. One suitable buffer, for example, is Tris-HCl pH8, NaCl100 mM and DTT 5 mM.

Screen development typically deploys a statistical approach to optimisation of bioassay parameters and includes a rigorous analysis of bioassay performance (in terms of signal:background ratio, intra-plate and inter-plate variability, etc) under optimised conditions. Appropriate quality control procedures may be built in to the final assay procedure to enable continuous assessment of performance during the running of screens. For example, the optimized assay described above may be transformed into a high throughput, semi-automated 384 well format in order to facilitate screening processes, such as screening of small molecule libraries.

Screening of Natural Product Libraries

In one embodiment, the present invention involves screening small molecule chemodiversity represented within libraries of parent and fractionated natural product extracts, to detect bioactive compounds as potential candidates for further characterization.

It is generally possible to test 100,000-250,000 samples during a primary screening phase. With hit rates frequently in the range of 0.1% -1%, the number of bioactive samples identified in a primary screen usually range from several hundred to several thousand. The process of primary screening may involve the use of specialised assay technologies, coupled with automated systems, which allow test sample throughputs of up to 50,000 per day. For example, the screening process may involve the use of a robotic screening system comprising a precise, six-axis robotic arm mounted on a linear track. Such as system links to all instrumentation and hardware, allowing microtiter plates to be transferred to any location on the system. Hardware includes plate carousels for storage and access of sample and assay plates, an automated system with robotic arm for liquid handling, a platewise microplate pipetting system, a plate shaker and a plate washer. This system also has a plate reader capable of fluorometric, photometric and luminometric detection. In addition to the robotic screening system, a number of stand-alone instruments for rapid microplate pipetting and for detection of a variety of signal read-outs from assay plates may be employed.

The process of dereplication may be used to select a small sub-population of hits identified in a primary screen that are most likely to contain active compounds with the desired characteristics. Experience has shown that dereplication is an important success-determining and rate-limiting step in natural products drug discovery.

Dereplication may be performed as follows. All hits from the initial PAC-1 inhibitor screen are subjected to a high-capacity fractionation procedure designed to generate information about the relative polarity of all active compounds present. Based on this information, all extracts displaying bioactivity in one or more of these initial fractions are progressed for HPLC separations using short gradients tailored to provide high resolution over the appropriate polarity ranges. With coupled UV/visible detection of eluates, testing of fractions for bioactivity both in the primary screen and relevant secondary assays, and analysis of active fractions by LC-MS, a package of physicochemical and bioactivity data on pure or nearly pure active HPLC fractions from all screen hits is generated.

Prioritized screen hits emerging from the de-replication process may be progressed for isolation and full chemical characterization of the active compounds present. In the case of microbial extracts, scaled-up quantities of the appropriate extracts may be first prepared by re-fermenting the producing organisms. In the case of plant tissue extracts, there are sufficient stocks of most of the dried and ground plant tissue specimens to prepare further quantities of extract for chemical isolation work.

In a preferred embodiment, the chemical isolation program aims to purify enough of each active compound to conduct structure elucidation work and further profiling of biological activity (typically 2-20 mg). Structures may be determined primarily on the basis of mass spectrometry (MS) and nuclear magnetic resonance (NMR) data.

Preferred bioassays developed as primary screens are also backed up by several secondary assays designed to detect false hits (eg due to interference with the assay detection system), hits due to unrelated modes of action (eg cytotoxicity in functional cell-based screens) or hits that fail to show the desired profile of biological specificity. Most secondary assays are reserved for use at a late stage in the dereplication process when they can be applied to pure or nearly pure active fractions derived from the hit extracts identified in primary screens. A number commercially available phosphatases, such as PP-1, PP-2A, PP-2B and PP-2C may be useful for this purpose. Dose response inhibition of lead hits identified in the PAC-1 assay are preferably also performed in order to measure potency and efficacy of inhibition.

Screening Protocol Based on Expression of PAC-1

An example of a screening method in which the ability of a candidate compound to inhibit PAC-1 expression may involve the following steps:
(i) contacting a candidate compound with cells capable of expressing PAC-1,
(ii) measuring the amount of expression of PAC-1 in the cells brought into contact with the candidate compound and comparing this amount of expression with the amount of expression (control amount of expression) of PAC-1 in the corresponding control cells not brought into contact with an investigational substance, and
(iii) selecting a candidate compound showing a reduced amount of expression of PAC-1 as compared with the amount of control expression on the basis of the result of the above step (ii).

The cells used in this screening method may be any cells that can express PAC-1, irrespective of the difference between natural and recombinant genes. Moreover, the derivation of the PAC-1 is not particularly limited. The cells may be human derived, or may derive from mammals other than humans such as mice, or from other organisms. Examples of suitable human cells are hematopoietic cells including mast cells. Moreover, transformed cells that contain expression vectors comprising nucleic acid sequences that encode PAC-1 may also be used.

The conditions for allowing the candidate compound to come into contact with the cells that can express PAC-1 are not limited, but it is preferable to select from among culture conditions (temperature, pH, culture composition, etc.) which will not kill the applicable cells, and in which the PAC-1 genes can be expressed.

The term “reduced” refers not only the comparison with the control amount of expression, but also encompasses cases where no PAC-1 is expressed at all. Specifically, this includes circumstances wherein the amount of expression of PAC-1 is substantially zero.

The amount of expression of PAC-1 can be assessed either by measuring the amount of expression of a PAC-1 gene (mRNA) or by measuring the amount of a PAC-1 protein produced. In addition, the method to measure the amount of PAC-1 need not be a method to directly measure the amount of expression of gene (mRNA) or the amount of protein produced, but may be any method that reflects these.

Specifically, to measure the amount of expression of PAC-1 (detection and assay), the amount of expression of PAC-1 mRNA may be measured utilizing DNA array or well-known methods such as the Northern blot method, as well as the RT-PCR method that utilizes oligonucleotides having nucleotide sequences complementary to the nucleotide sequence of the applicable PAC-1 mRNA. Moreover, the amount of PAC-1 protein may be measured by implementing such well-known methods as the Western blot method utilizing an anti-PAC-1 antibody.

The measurement of the amount of expression of PAC-1 (detection and assay) may be implemented by measuring the activity of proteins derived from marker genes, using a cell line into which have been introduced fused genes comprising the marker genes such as reporter genes (e.g., luciferase genes, chloramphenicol-acetyltransferase genes, β-glucuronidase genes, β-galactosidase genes and aequorin genes) linked to the suppression region of the PAC-1 gene.

Screening Protocol Based on Binding of PAC-1 to One or More of its Binding Partners

In one embodiment, PAC-1 agonists or antagonists are identified by screening for candidate compounds which interfere with the binding of PAC-1 to a PAC-1 substrate. Examples of PAC-1 substrates include ERK, p38 and JNK.

Standard solid-phase ELISA assay formats are particularly useful for identifying antagonists of the protein-protein interaction. In accordance with this embodiment, one of the binding partners, e.g a PAC-1 substrate (such as ERK, p38 or JNK or a portion thereof) is immobilized on a solid matrix, such as, for example an array of polymeric pins or a glass support. Conveniently, the immobilized binding partner is a fusion polypeptide comprising Glutathione-S-transferase (GST; e.g. a CAP-ERK fusion), wherein the GST moiety facilitates immobilization of the protein to the solid phase support. The second binding partner (e.g. PAC-1) in solution is brought into physical relation with the immobilized protein to form a protein complex, which complex is detected using antibodies directed against the second binding partner. The antibodies are generally labelled with fluorescent molecules or conjugated to an enzyme (e.g. horseradish peroxidase), or alternatively, a second labelled antibody can be used that binds to the first antibody. Conveniently, the second binding partner is expressed as a fusion polypeptide with a FLAG or oligo-histidine peptide tag, or other suitable immunogenic peptide, wherein antibodies against the peptide tag are used to detect the binding partner. Alternatively, oligo-HIS tagged protein complexes can be detected by their binding to nickel-NTA resin (Qiagen), or FLAG-labeled protein complexes detected by their binding to FLAG M2 Affinity Gel (Kodak). It will be apparent to the skilled person that the assay format described herein is amenable to high throughput screening of samples, such as, for example, using a microarray of bound peptides or fusion proteins.

A two-hybrid assay as described in U.S. Pat. No. 6,316,223 may also be used to identify compounds that interfere with the binding of PAC-1 to one of its substrates. The basic mechanism of this system is similar to the yeast two hybrid system. In the two-hybrid system, the binding partners are expressed as two distinct fusion proteins in a mammalian host cell. In adapting the standard two-hybrid screen to the present purpose, a first fusion protein consists of a DNA binding domain which is fused to one of the binding partners, and a second fusion protein consists of a transcriptional activation domain fused to the other binding partner. The DNA binding domain binds to an operator sequence which controls expression of one or more reporter genes. The transcriptional activation domain is recruited to the promoter through the functional interaction between binding partners. Subsequently, the transcriptional activation domain interacts with the basal transcription machinery of the cell, thereby activating expression of the reporter gene(s), the expression of which can be determined. Candidate bioactive agents that modulate the protein-protein interaction between the binding partners are identified by their ability to modulate transcription of the reporter gene(s) when incubated with the host cell. Antagonists will prevent or reduce reporter gene expression, while agonists will enhance reporter gene expression. In the case of small molecule modulators, these are added directly to the cell medium and reporter gene expression determined. On the other hand, peptide modulators are expressible from nucleic acid that is transfected into the host cell and reporter gene expression determined. In fact, whole peptide libraries can be screened in transfected cells.

Alternatively, reverse two hybrid screens, such as, for example, described by Vidal et al., Proc. Natl Acad. Sci USA 93, 10315-10320, 1996, may be employed to identify antagonist molecules. Reverse hybrid screens differ from froward screens supra in so far as they employ a counter-selectable reporter gene, such as for example, CYH2 or LYS2, to select against the protein-protein interaction. Cell survival or growth is reduced or prevented in the presence of a non-toxic substrate of the counter-selectable reporter gene product, which is converted by said gene product to a toxic compound. Accordingly, cells in which the protein-protein interaction of the invention does not occur, such as in the presence of an antagonist of said interaction, survive in the presence of the substrate, because it will not be converted to the toxic product. For example, a portion/fragment of PAC-1 that binds to ERK or p38 or JNK is expressed as a DNA binding domain fusion, such as with the DNA binding domain of GAL4; and the portion of ERK or p38 or JNK that binds PAC-1 is expressed as an appropriate transcription activation domain fusion polypeptide (e.g. with the GAL4 transcription activation domain). The fusion polypeptides are expressed in yeast in operable connection with the URA3 counter-selectable reporter gene, wherein expression of URA3 requires a physical relation between the GALA DNA binding domain and transcriptional activation domain. This physical relation is achieved, for example, by placing reporter gene expression under the control of a promoter comprising nucleotide sequences to which GAL4 binds. Cells in which the reporter gene is expressed do not grow in the presence of uracil and 5-fluororotic acid (5-FOA), because the 5-FOA is converted to a toxic compound. Candidate peptide inhibitor(s) are expressed in libraries of such cells, wherein cells that grow in the presence of uracil and 5-FOA are retained for further analysis, such as, for example, analysis of the nucleic acid encoding the candidate peptide inhibitor(s). Small molecules that antagonize the interaction are determined by incubating the cells in the presence of the small molecules and selecting cells that grow or survive of cells in the presence of uracil and 5-FOA.

Alternatively, a protein recruitment system, such as that described in U.S. Pat. No. 5,776,689 to Karin et al., may be used. In a standard protein recruitment system, a protein-protein interaction is detected in a cell by the recruitment of an effector protein, which is not a transcription factor, to a specific cell compartment. Upon translocation of the effector protein to the cell compartment, the effector protein activates a reporter molecule present in that compartment, wherein activation of the reporter molecule is detectable, for example, by cell viability, indicating the presence of a protein-protein interaction.

More specifically, the components of a protein recruitment system include a first expressible nucleic acid encoding a first fusion protein comprising the effector protein and one of the binding partners (e.g. ERK or p38 or JNK or a portion thereof), and a second expressible nucleic acid molecule encoding a second fusion protein comprising a cell compartment localization domain and the other binding partner (e.g. PAC-1 or a portion thereof). A cell line or cell strain in which the activity of an endogenous effector protein is defective or absent (e.g. a yeast cell or other non-mammalian cell), is also required, so that, in the absence of the protein-protein interaction, the reporter molecule is not expressed.

A complex is formed between the fusion polypeptides as a consequence of the interaction between the binding partners, thereby directing translocation of the complex to the appropriate cell compartment mediated by the cell compartment localization domain (e.g. plasma membrane localization domain, nuclear localization domain, mitochondrial membrane localization domain, and the like), where the effector protein then activates the reporter molecule. Such a protein recruitment system can be practiced in essentially any type of cell, including, for example, mammalian, avian, insect and bacterial cells, and using various effector protein/reporter molecule systems.

For example, a yeast cell based assay is performed, in which the interaction between PAC-1 and one or more of its binding partners results in the recruitment of a guanine nucleotide exchange factor (GEF or C3G) to the plasma membrane, wherein GEF or C3G activates a reporter molecule, such as Ras, thereby resulting in the survival of cells that otherwise would not survive under the particular cell culture conditions. Suitable cells for this purpose include, for example, Saccharomyces cerevisiae cdc25-2 cells, which grow at 36° C. only when a functional GEF is expressed therein, Petitjean et al., Genetics 124, 797-806, 1990) Translocation of the GEF to the plasma membrane is facilitated by a plasma membrane localization domain. Activation of Ras is detected, for example, by measuring cyclic AMP levels in the cells using commercially available assay kits and/or reagents. To detect antagonists of the protein-protein interaction of the present invention, duplicate incubations are carried out in the presence and absence of a test compound, or in the presence or absence of expression of a candidate antagonist peptide in the cell. Reduced survival or growth of cells in the presence of a candidate compound, or candidate peptide indicates that the peptide or compound is an antagonist of the interaction between PAC-1 and one or more of its binding partners.

A “reverse” protein recruitment system is also contemplated, wherein modified survival or modified growth of the cells is contingent on the disruption of the protein-protein interaction by the candidate compound or candidate peptide. For example, NIH 3T3 cells that constitutively express activated Ras in the presence of GEF can be used, wherein the absence of cell transformation is indicative of disruption of the protein complex by a candidate compound or peptide. In contrast, NIH 3T3 cells that constitutively express activated Ras in the presence of GEF have a transformed phenotype (Aronheim et al., Cell. 78, 949-961, 1994)

In yet another embodiment, small molecules are tested for their ability to interfere with binding of PAC-1 to one of its substrates, by an adaptation of plate agar diffusion assay described by Vidal and Endoh, TIBS 17, 374-381, 1999, which is incorporated herein by reference.

Models of Inflammation

In vivo models of inflammation are available which can be used to assess the efficacy of PAC-1 agonists or antagonists identified as described above as therapeutic agents. For example, the suppressive effect (prophylactic efficacy, therapeutic efficacy) of the selected candidate compound in rheumatoid arthritis can be evaluated by a pharmacological efficacy test using an animal (e.g. mouse) model of collagen-induced arthritis (Trentham et al., J Exp Med 146: 857 (1977)), K/B×N serum-induced arthritis (Kouskoff et al., Cell 87: 811 (1996)), antigen-induced arthritis, or adjuvant-induced arthritis (Pearson, Proc Soc. Exp. Biblo. Med. 91:. 95 (1956)).

Further examples of suitable animal models include: cecal ligation puncture (CLP) model of sepsis (Huber-Lang, M. S., et al. (2002) Faseb J 16(12): 1567-74); rat model of RA (Woodruff, T. M., et al. (2002) Arthritis Rheum 46(9): 2476-85); porcine model of sepsis (Mohr, M., et al. (1998) Eur J Clin Invest 28(3): 227-34); immune complex-induced lung disease; pancreatitis associated lung injury (Bhatia, M., et al. (2001) Am J Physiol Gastrointest Liver Physiol 280(5): G974-8); acute lung injury; renal ischaemia-reperfusion injury; collagen-induced arthritis; and experimental airway disease (asthma like model).

In another example, leukocyte infiltration upon intradermal injection of a candidate compound can be monitored (see e.g., Van Damme, J. et al., J. Exp. Med., 176: 59-65 (1992); Zachariae, C. 0. C. et al., J. Exp. Med. 171: 2177-2182 (1990); Jose, P. J. et al., J. Exp. Med. 179: 881-887 (1994)). In one embodiment, skin biopsies are assessed histologically for infiltration of leukocytes (e.g., eosinophils, granulocytes). A decrease of the extent of infiltration in the presence of the candidate compound as compared with the extent of infiltration in the absence of the candidate compound is indicative of anti-inlammatory properties.

The candidate compounds validated in these model systems can be further subjected to structural analysis and, based on the result, can be produced on a commercial scale by chemical synthesis, biological synthesis (fermentation) or a genetic procedure.

Therapeutic Methods

The PAC-1 agonists or antagonists identified by the methods of the present invention can be used therapeutically for inflammatory diseases. The term “therapeutically” or as used herein in conjunction with the PAC-1 agonists or antagonists of the invention denotes both prophylactic as well as therapeutic administration. Thus, PAC-1 agonists/antagonists can be administered to high-risk patients in order to lessen the likelihood and/or severity of an inflammatory disease or administered to patients already evidencing active disease, for example rheumatoid arthritis.

The selected PAC-1 inhibitors can be used to treat allergy, atherogenesis, anaphylaxis, malignancy, chronic and acute inflammation, histamine and IgE-mediated allergic reactions, shock, and rheumatoid arthritis, atherosclerosis, multiple sclerosis, allograft rejection, fibrotic disease, asthma, inflammatory glomerulopathies or any immune complex related disorder.

Diseases or conditions of humans or other species which can be treated with inhibitors of PAC-1 function include, but are not limited to:

(a) inflammatory or allergic diseases and conditions, including respiratory allergic diseases such as asthma, allergic rhinitis, hypersensitivity lung diseases, hypersensitivity pneumonitis, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); anaphylaxis or hypersensitivity responses, drug allergies (e.g., to penicillin, cephalosporins), insect sting allergies; inflammatory bowel diseases, such as Crohn's disease and ulcerative colitis; spondyloarthropathies; scleroderma; psoriasis and inflammatory dermatoses such as dermatitis, eczema, atopic dermatitis, allergic contact dermatitis, urticaria; vasculitis (e.g., necrotizing, cutaneous, and hypersensitivity vasculitis);

(b) autoimmune diseases, such as arthritis (e.g., rheumatoid arthritis, psoriatic arthritis), multiple sclerosis, systemic lupus erythematosus, myasthenia gravis, juvenile onset diabetes, nephritides such as glomerulonephritis, autoimmune thyroiditis, Behcet's disease;

(c) graft rejection (e.g., in transplantation), including allograft rejection or graft-versus-host disease;

(d) atherosclerosis;

(e) cancers with leukocyte infiltration of the skin or organs;

(f) other diseases or conditions, in which undesirable inflammatory responses are to be inhibited can be treated, including, but not limited to, reperfusion injury, stroke, adult respiratory distress syndrome, certain hematologic malignancies, cytokine-induced toxicity (e.g., septic shock, endotoxic shock), polymyositis, dermatomyositis, pemphigoid, Alzheimers Disease and granulomatous diseases including sarcoidosis.

Diseases or conditions of humans or other species which can be treated with promoters of PAC-1 function, include, but are not limited to immunosuppression, such as that in individuals with immunodeficiency syndromes such as AIDS, individuals undergoing radiation therapy, chemotherapy, therapy for autoimmune disease or other drug therapy (e.g., corticosteroid therapy), which causes immunosuppression; and immunosuppression due congenital deficiency in receptor function or other causes.

Modes of Administration

In the case where the candidate compound is in the form of a low molecular weight compound, a peptide or a protein such as an antibody, the substance can be formulated into the ordinary pharmaceutical compositions (pharmaceutical preparations) which are generally used for such forms, and such compositions can be administered orally or parenterally. Generally speaking, the following dosage forms and methods of administration can be utilized

The dosage form includes such representative forms as solid preparations, e.g. tablets, pills, powders, fine powders, granules, and capsules, and liquid preparations, e.g. solutions, suspensions, emulsions, syrups, and elixirs. These forms can be classified by the route of administration into said oral dosage forms or various parenteral dosage forms such as transnasal preparations, transdermal preparations, rectal preparations (suppositories), sublingual preparations, vaginal preparations, injections (intravenous, intraarterial, intramuscular, subcutaneous, intradermal) and drip injections. The oral preparations., for instance, may for example be tablets, pills, powders, fine powders, granules, capsules, solutions, suspensions, emulsions, syrups, etc. and the rectal and vaginal preparations include tablets, pills, and capsules, among others. The transdermal preparations may not only be liquid preparations, such as lotions, but also be semi-solid preparations, such as reams, ointments, and so forth.

The injections may be made available in such forms as solutions, suspensions and emulsions, and as vehicles, sterilized water, water-propylene glycol, buffer solutions, and saline of 0.4 weight % concentration can be mentioned as examples. These injections, in such liquid forms, may be frozen or lyophilized. The latter products, obtained by lyophilization, are extemporaneously reconstituted with distilled water for injection or the like and administered. The above forms of pharmaceutical composition (pharmaceutical preparation) can be prepared by formulating the compound having PAC-1 inhibitory action and a pharmaceutically acceptable carrier in the manner established in the art. The pharmaceutically acceptable carrier includes various excipients, diluents, fillers, extenders, binders, disintegrators, wetting agents, lubricants, and dispersants, among others. Other additives which are commonly used in the art can also be formulated. Depending on the form of pharmaceutical composition to be produced, such additives can be judiciously selected from among various stabilizers, fungicides, buffers, thickeners, pH control agents, emulsifiers, suspending agents, antiseptics, flavors, colors, tonicity control or isotonizing agents, chelating agents and surfactants, among others.

The pharmaceutical composition in any of such forms can be administered by a route suited to the objective disease, target organ, and other factors. For example, it may be administered intravenously, intraarterially, subcutaneously, intradermally, intramuscularly or via airways. It may also be directly administered topically into the affected tissue or even orally or rectally.

The dosage and dosing schedule of such a pharmaceutical preparation vary with the dosage form, the disease or its symptoms, and the patient's age and body weight, among other factors, and cannot be stated in general terms. The usual dosage, in terms of the daily amount of the active ingredient for an adult human, may range from about 0.0001 mg to about 500 mg, preferably about 0.001 mg.about.about 100 mg, and this amount can be administered once a day or in a few divided doses daily.

When the substance having PAC- 1 inhibitory activity is in the form of a polynucleotide such as an antisense compound, the composition may be provided in the form of a drug for gene therapy or a prophylactic drug. Recent years have witnessed a number of reports on the use of various genes, and gene therapy is by now an established technique.

The drug for gene therapy can be prepared by introducing the object polynucleotide into a vector or transfecting appropriate cells with the vector. The modality of administration to a patient is roughly divided into two modes, viz. The mode applicable to (1) the case in which a non-viral vector is used and the mode applicable to (2) the case in which a viral vector is used. Regarding the case in which a viral vector is used as said vector and the case in which a non-viral vector is used, respectively, both the method of preparing a drug for gene therapy and the method of administration are dealt with in detail in several books relating to experimental protocols [e.g. “Bessatsu Jikken Igaku, Idenshi Chiryo-no-Kosogijutsu (Supplement to Experimental Medicine, Fundamental Techniques of Gene Therapy), Yodosha, 1996; Bessatsu Jikken Igaku: Idenshi Donyu & Hatsugen Kaiseki Jikken-ho (Supplement to Experimental Medicine: Experimental Protocols for Gene Transfer & Expression Analysis), Yodosha, 1997; Japanese Society for Gene Therapy (ed.): Idenshi Chiryo Kaihatsu Kenkyn Handbook (Research Handbook for Development of Gene Therapies), NTS, 1999, etc.].

When using a non-viral vector, any expression vector capable of expressing the anti-PAC-1 nucleic acid may be used. Suitable examples include pCAGGS (Gene 108: 193 (1991)), pBK-CMV, pcDNA 3.1, and pZeoSV (Invitrogen, Stratagene).

Transfer of a polynucleotide into the patient can be achieved by inserting the object polynucleotide into such a non-viral vector (expression vector) in the routine manner and administering the resulting recombinant expression vector. By so doing, the object polynucleotide can be introduced into the patient's cells or tissue.

More particularly, the method of introducing the polynucleotide into cells includes the calcium phosphate transfection (coprecipitation) technique and the DNA (polynucleotide) direct injection method using a glass microtube, among others.

The method of introducing a polynucleotide into a tissue includes the polynucleotide transfer technique using internal type liposomes or electrostatic type liposomes, the HVJ-liposome technique, the modified HVJ-liposome (HVJ-AVE liposome) technique, the receptor-mediated polynucleotide transfer technique, the technique which comprises transferring the polynucleotide along with a vehicle (metal particles) into cells with a particle gun, the naked-DNA direct transfer technique, and the transfer technique using a positively charged polymer, among others.

Suitable viral vectors include vectors derived from recombinant adenoviruses and retrovirus. Examples include vectors derived from DNA or RNA viruses such as detoxicated retrovirus, adenovirus, adeno-associated virus, herpesvirus, vaccinla virus, poxvirus, poliovirus, sindbis virus, Sendai virus, SV40, human immunodeficiency virus (H) and so forth. The adenovirus vector, in particular, is known to be by far higher in infection efficiency than other viral vectors and, from this point of view, the adenovirus vector is preferably used.

Transfer of the polynucleotide into the patient can be achieved by introducing the object polynucleotide into such a viral vector and infecting the desired cells with the recombinant virus obtained. In this manner, the object polynucleotide can be introduced into the cells.

The method of administering the thus-prepared drug for gene therapy to the patient includes the in vivo technique for introducing the drug for gene therapy directly into the body and the ex vivo technique which comprises withdrawing certain cells from a human body, introducing the drug for gene therapy into the cells in vitro and returning the cells into the human body (Nikkei Science, April, 1994 issue, 20-45;

Pharmaceuticals Monthly, 36(1), 23-48, 1994; Supplement to Experimental Medicine, 12(15), 1994; Japanese Society for Gene Therapy (ed.): Research Handbook for Development of Gene Therapies, NTS, 1991). For use in the prevention or treatment of an inflammatory disease to which the present invention is addressed, the drug is preferably introduced into the body by the in vivo technique.

When the in vivo method is used, the drug can be administered by a route suited to the object disease, target organ or the like. For example, it can be administered intravenously, intraarterially, subcutaneously or intramuscularly, for instance, or may be directly administered topically into the affected tissue.

The drug for gene therapy can be provided in a variety of pharmaceutical forms according to said routes of administration. In the case of an injectable form, for instance, an injection can be prepared by the per se established procedure, for example by dissolving the active ingredient polynucleotide in a solvent, such as a buffer solution, e.g. PBS, physiological saline, or sterile water, followed by sterilizing through a filter where necessary, and filling the solution into sterile vitals, Where necessary, this injection may be supplemented with the ordinary carrier or the like. In the case of liposomes such as HVJ-liposome, the drug can be provided in various liposome-entrapped preparations in such forms as suspensions, frozen preparations and centrifugally concentrated frozen preparations.

Furthermore, in order that the gene may be easily localized in the neighborhood of the affected site, a sustained-release preparation (eg. a minipellet) may be prepared and implanted near the affected site or the drug may be administered continuously and gradually to the affected site by means of an osmotic pump or the like.

The polynucleotide content of the drug for gene therapy can be judiciously adjusted according to the disease to be treated, the patient's age and body weight, and other factors but the usual dosage in terms of each polynucleotide is about 0.0001 to about 100 mg, preferably about 0.001 to about 10 mg. This amount is preferably administered several days or a few months apart.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

The present invention will now be illustrated by the following Examples, which are not intended to be limiting in any way. The teachings of all references cited herein are incorporated herein by reference.

### EXAMPLE 1

### Microarray Analysis of PAC-1 Expression

Methods

Preparation of cRNA and Genechip hybridisations: Depending on the quantity of RNA available cRNA was prepared using the GeneChip Expression Analysis Technical Manual (Affymetrix, Santa Clara, Calif.) or the cRNA methods published in Baugh et al (Nucl. Acids Res. 29: E29 (2001)). The GeneChip Expression Analysis protocol involved cDNA synthesis from 20 ug of total RNA using a poly(T) primer containing a T7 RNA polymerase promoter GGC CAG TGA ATT GTA ATA CGA CTC ACT ATA GGG AGG CGG-(dT)24 (Geneworks, Australia) (SEQ ID NO: 86). cRNA was transcribed from cDNA and biotinylated using the BioArray High Yield RNA Transcript Labelling Kit (Enzo Diagnostics, Farmingdale, N.Y.). Twenty micrograms of cRNA was fragmented by heating at 94° C. for 35 min in fragmentation buffer (40 mM Tris-acetate (pH 8.1), 125 mM KOAc, 30 mM MgOAc) prior to hybridisation.

For the Baugh et al (2001, supra) amplification, cDNA synthesis volumes were different from the GeneChip Expression Analysis Technical Manual but reaction component concentrations, incubation times and temperatures were conserved. It should be noted that only 500 ng of starting RNA was used; T4gp32 and RNAse Inhibitor were incorporated in the 1st cDNA synthesis reaction to give 2.4 mg and 20 u respectively, enzymes were heat inactivated at 70° C. instead of using EDTA, the cDNA phenol-chloroform extraction was followed by chromatography on a BioGel p-6 column (Bio-Rad) and an ethanol precipitation without NH4Oac. Fifteen micrograms of cRNA was fragmented prior to hybridisation.

Hybridisation cocktails were then made by adding fragmented cRNA, control cRNAs, grid alignment oligonucleotides and blocking reagents. These mixtures were hybridised overnight (˜16 h) to individual Test3 (Affymetrix) arrays at 45° C., under constant rotation at 60 rpm. Washing and staining of the hybridised arrays were performed by an Affymetix Fluidics Station, according to the manufacturers protocols. Fluorescent signals were measured on the arrays using the Agilent GeneArray Laser Scanner and gene transcript levels were determined and scaled to 150 using alogorithms in MicroArray Analysis Suite Software 5.0 (Affymetrix). Hybridisation cocktails that met the test 3 criteria, background less then 150, GAPDH and B-actin 3′/5′ ratios less then 3 and similar scaling factors between samples, were then run on U95A arrays. Relative mRNA expression levels on the IL4 and IL13 stimulated NHBE arrays were expressed as plus or minus fold changes when compared to the control NHBE array. Genes that showed a change of 2 fold or greater in at least two separate experiments were considered differentially expressed.

Results

Table 1 shows the results obtained using the following single microarray Gene Chips:
OA control Synovial tissue was obtained from Osteoarthritis (OA) patients undergoing surgery at St Vincent's Hospital, Sydney, Australia. This tissue was used to establish fibroblast-like synoviocyte cultures and gene expression was examined.
OA TNF Synoviocytes from osteoarthritis (OA) patients were stimulated with 10 ng/ml of the cytokine Tumour Necrosis Factor (TNF)-α for 4 hours at 37° C. and gene expression was examined.
RA control Synovial tissue was obtained from Rheumatoid Arthritis (RA) patients undergoing surgery at St Vincent's Hospital, Sydney, Australia. This tissue was used to establish fibroblast-like synoviocyte cultures and gene expression was examined.
RA IL-1 Synoviocytes from Rheumatoid Arthritis (RA) patients were stimulated with 10 ng/ml of the cytokine Interleukin (IL)-β for 4 hours at 37° C. and gene expression was examined.
RA TNF Synoviocytes from Rheumatoid Arthritis (RA) patients were stimulated with 10 ng/ml of the cytokine Tumour Necrosis Factor (TNF)-α for 4 hours at 37° C. and gene expression was examined.
HMC1 HMC1 is an immature human mast cell line derived from a leukemia patient.
Alpha4beta7 α4β7, an integrin adhesion molecule is a marker for gut homing effector memory T cells. These cells were isolated from human peripheral blood using cell sorting and gene expression examined.
BSCM cont Bronchial Smooth Muscle Cells (BSMCs) were obtained commercially from Clonetics (San Diego, Calif.) and gene expression examined.
BSCM IL4 Bronchial Smooth Muscle Cells (BSMCs) were obtained commercially from Clonetics (San Diego, Calif.) and activated with 10 ng/ml of interleukin (IL)-4 for 18 hours at 37° C.
BSCM IL13 Bronchial Smooth Muscle Cells (BSMCs) were obtained commercially from Clonetics (San Diego, Calif.) and activated with 10 ng/ml of interleukin (IL)-13 for 18 hours at 37° C.
CLA Cutaneous Lymphocyte Antigen (CLA) is a marker for skin homing effector memory T cells. These cells were isolated from human peripheral blood using cell sorting.
MC control Mast cells were derived from human cord blood using a ficoll density gradient and differentiated to mature mast cells over 6-9 weeks using 100 ng/ml stem cell factor, 10 ng/ml interleukin-10 (IL10), and 5 ng/ml IL-6. Gene expression was then examined.
MC anti-IgE
Wk6 Mast cells were derived from human cord blood using a ficoll density gradient and differentiated to mature mast cells over 6-9 weeks using 100 ng/ml stem cell factor, 10 ng/ml interleukin-10 (IL-10), and 5 ng/ml IL-6. Once mature, cells were first primed with 4 μg/ml human IgE anti-NP for 18 hours and then activated with 5 μg/ml mouse anti-human IgE for 2 hours by crosslinking the IgE receptors.
NHBE 18 hr
Control NHBE primary cell lines were purchased from Clonetics (San Diego, Calif.) and were used to represent human lung epithelial cell behaviour in response to Th2 cytokines IL4 and IL13. Both NHBE cell lines, lot 8F1142 and 7F1482, were isolated from Caucasian males aged 18 months and 32 years respectively.
NHBE cells were maintained in Clonetics bronchial epithelial growth media (BEGM), which included supplements of 52 mg/l bovine pituitary extract, 0.5 mg/l hydrocortisone, 0.5 mg/l human recombinant epidermal growth factor, 0.5 mg/l epinephrine, 10 mg/l transferrin, 5 mg/l insulin, 0.1 mg/l retinoic acid, 6.5 mg/l Triiodothryonine, 50 mg/l gentamicin, and 50 mg/I amphotericin B (Clonetics). Media was replaced every 3 to 4 days. When confluent, cells were subcultured at a ratio of 1:3, 0.025% trypsin-EDTA (Gibco) was used to dislodge cells and 100% foetal bovine serum for neutralisation (Gibco).
NHBE 18 hr
IL13 Normal Human Bronchial Epithelial (NHBE) cells stimulated with 10 ng/ml of interleukin (IL)-13 for 18 hours at 37° C.
NHBE 18 hr
IL 4 Normal Human Bronchial Epithelial (NHBE) cells stimulated with 10 ng/ml of interleukin (IL)-4 for 18 hours at 37° C.
CCR7+ CCR7+ (CD4+, CD45RO+) represent Central Memory T cells and were isolated from human peripheral blood using cell sorting techniques.
CCR7− CCR7− (CD4+, CD45RO+) represent Effector Memory T cells and were isolated from human peripheral blood using cell sorting.
CD57+ CD57+ (CXCR5+, CD4+) represent T Follicular Homing cells and were isolated from human tonsil tissue using cell sorting.
CD57− CD57− (CXCR5+, CD4+) are not T Follicular Homing cells and were isolated from human tonsil tissue using cell sorting.
CD8+
CCR7−
RO+ Cytotoxic effector memory (CD8+, CCR7-, RO-) were isolated from human peripheral blood using cell sorting.
CD8+
CCR7−
RO− Cytotoxic terminally differentiated, T cells (CD8+, CCR7−, RO+) were isolated from human peripheral blood using cell sorting.
TH1 human CD4+ T cells were isolated from human umbilical cord blood and polarised in vitro using IL-12 and neutralising IL-4.
TH2 human CD4+ T cells were isolated from human umbilical cord blood and polarised in vitro using IL-4 and neutralising IL-12 and interferon γ.
Control
Eosinophils Eosinophils were isolated from human peripheral blood using a percoll gradient method (Hansel et al., 1989, J Immunol Methods 122 97-103) with modifications.
2hr
eosinophils Eosinophils were isolated from human peripheral blood using a Percoll gradient method and stimulated with 50 ng/ml of Phorbol-12-myristate-13-acetate (PMA) for 2 hours at 37° C.
Week 4
Mast cell Mast cells were derived from human cord blood using a ficoll density gradient and differentiated to mature mast cells over 4 weeks using 100 ng/ml stem cell factor, 10 ng/ml interleukin-10 (IL-10), and 5 ng/ml IL 6. Gene expression was then examined.
Week 9
Mast cell Mast cells were derived from human cord blood using a ficoll density gradient and differentiated to mature mast cells over 9 weeks using 100 ng/ml stem cell factor, 10 ng/ml interleukin-10 (IL-10), and 5 ng/ml IL-6. Gene expression was then examined.
   Table 2 shows the results obtained using the following microarray comparison Gene Chips:
   GeneChip-HuFL2
OA cont vs RA cont Synovial tissue was obtained from Osteoarthritis (OA) and Rheumatoid Arthritis (RA) patients undergoing surgery at St Vincent's Hospital, Sydney, Australia. This tissue was used to establish fibroblast-like synoviocyte cultures. The cultures used for GeneChip studies were derived from biopsies taken from two knee biopsy samples from 37 and 38 year old women. This GeneChip compared gene expression of unstimulated synoviocyte cultures from OA and RA patients.
OA TNF vs OA control Synoviocytes from osteoarthritis (OA) patients were stimulated with 10 ng/ml of the cytokine Tumour Necrosis Factor (TNF)-α (for 4 hours at 37° C. This GeneChip compared gene expression of unstimulated synoviocyte cultures from OA patients to those stimulated with TNF-α.
OA TNF vs RA TNF This GeneChip compared synoviocyte cultures from OA patients that were stimulated with TNF-α to synoviocytes cultures from RA patients that were stimulated with TNF-α.
RA IL-1 vs RA cont Synoviocytes from RA patients were stimulated with 10 ng/ml of the cytokine interleukin (IL)-1 β for 4 hours at 37° C. This GeneChip compared gene expression of unstimulated synoviocyte cultures from RA patients to those stimulated with IL-1β.
RA TNF vs RA IL-1β This GeneChip compared gene expression of TNF-α stimulated synoviocytes to IL-1β stimulated synoviocytes from RA patients.
BSMC IL-13 vs cont Bronchial Smooth Muscle Cells (BSMCs) were obtained commercially from Clonetics (San Diego, Calif.) and stimulated with 10 ng/ml of interleukin (IL)-13 for 18 hours at 37° C. This GeneChip compared gene expression of IL-13 stimulated BSMCs to unstimulated BSMCs.
   GeneChip-U95
CLA vs α4β7 Cutaneous Lymphocyte Antigen (CLA) is a marker for skin homing effector memory T cells and α4β7, an integrin adhesion molecule is a marker for gut homing effector memory T cells. These cells were isolated from human peripheral blood using cell sorting. This Gene Chip compares gene expression in skin homing (CLA) T cells to gut homing (α4β7) T cells.
NHBE IL-13 vs cont Normal Human Bronchial Epithelial (NHBE) cells were obtained commercially from Clonetics (San Diego, Calif.) and stimulated with 10 ng/ml of interleukin (IL)-13 for 18 hours at 37° C. This GeneChip compared gene expression of IL-13 stimulated NHBEs to unstimulated NHBEs.
NHBE IL-4 vs cont Normal Human Bronchial Epithelial (NHBE) cells were obtained commercially from Clonetics (San Diego, Calif.) and stimulated with 10 ng/ml of interleukin (IL)-4 for 18 hours at 37° C. This GeneChip compared gene expression of IL-4 stimulated NHBEs to unstimulated NHBEs.
TNF RA vs cont RA Synoviocytes from Rheumatoid Arthritis (RA) patients were stimulated with 10 ng/ml of the cytokine Tumour Necrosis Factor (TNF)-α for 4 hours at 37° C. This GeneChip compared gene expression of synoviocyte cultures from RA patients stimulated with TNF-α to unstimulated RA synoviocytes.
RA IL-1 vs RA cont Synoviocytes from RA patients were stimulated with 10 ng/ml of the cytokine interleukin (IL)-1β for 4 hours at 37° C. This GeneChip compared gene expression of unstimulated synoviocyte cultures from RA patients to those stimulated with IL-1β.
RA IL-4 vs RA cont Synoviocytes from RA patients were stimulated with 10 ng/ml of the cytokine interleukin (IL)4 for 4 hours at 37° C. This GeneChip compared gene expression of unstimulated synoviocyte cultures from RA patients to those stimulated with IL-4.
RA IL-1β vs cont Synoviocytes from RA patients were stimulated with 10 ng/ml of the cytokine interleukin (IL)-1β for 4 hours at 37° C. This GeneChip compared gene expression of unstimulated synoviocyte cultures from RA patients to those stimulated with IL-1β.
MC anti-IgE Wk6 vs cont Mast cells were derived from human cord blood using a ficoll density gradient and differentiated to mature mast cells over 6 weeks using 100 ng/ml stem cell factor, 10 ng/ml interleukin-10 (IL-10), and 5 ng/ml IL-6. Once mature, cells were first primed with 4 μg/ml human IgE anti-NP for 18 hours and then activated with 5 μg/ml mouse anti-human IgE for 2 hours by crosslinking the IgE receptors. This GeneChip compared gene expression of unstimulated mast cells to those stimulated with IgE.
BSCM IL4 vs cont Bronchial Smooth Muscle Cells (BSMCs) were obtained commercially from Clonetics (San Diego, Calif.) and stimulated with 10 ng/ml of interleukin (IL)-4 for 18 hours at 37° C. This GeneChip compared gene expression of IL-4 stimulated BSMCs to unstimulated BSMCs.
BSCM IL-13 vs cont Bronchial Smooth Muscle Cells (BSMCs) were obtained commercially from Clonetics (San Diego, Calif.) and stimulated with 10 ng/ml of interleukin (IL)-13 for 18 hours at 37° C. This GeneChip compared gene expression of IL-13 stimulated BSMCs to unstimulated BSMCs.
   GeneChip-U133
CCR7+ vs CCR7− CCR7+ (CD4+, CD45RO+) representing Central Memory T cells and CCR7− (CD4+, CD45RO+) representing Effector Memory T cells were isolated from human peripheral blood using cell sorting. This GeneChip compares gene expression in the Central Memory T cell subset (CCR7+) to the Effector Memory T cell subset (CCR7−).
CD57+ vs CD57− CD57+(CXCR5+, CD4+) representing T Follicular Homing cells and CD57− (CXCR5+, CD4+) were isolated from human tonsil tissue using cell sorting. This GeneChip compares gene expression in T follicular homing cell subset (CD57+) to non T follicular homing cells (CD57−).
CD8+CCR7−RO− vs CD8+CCR7−RO+ Cytotoxic effector memory (CD8+, CCR7−, RO−) and cytotoxic terminally differentiated T cells (CD8+, CCR7−, RO+) were isolated from human peripheral blood using cell sorting. This GeneChip compares gene expression in cytotoxic effector memory T cells (RO−) to cytotoxic terminally differentiated (RO+) T cells.
Wk9 vs Wk 4 MCs Mast cells were derived from human cord blood using a ficoll density gradient and differentiated to mature mast cells over time using 100 ng/ml stem cell factor, 10 ng/ml interleukin-10 (IL-10), and 5 ng/ml IL-6. This GeneChip compares gene expression of 4 week-old mast cells to 9 week-old mast cells.
IgE vs cont MC Mast cells were derived from human cord blood using a ficoll density gradient and differentiated to mature mast cells over 7 weeks using 100 ng/ml stem cell factor, 10 ng/ml interleukin-10 (IL-10), and 5 ng/ml IL-6. Once mature, cells were first primed with 4 μg/ml human IgE anti-NP for 18 hours and then activated with 5 μg/ml mouse anti-human IgE for 2 hours by crosslinking the IgE receptors. This GeneChip compared gene expression of unstimulated mast cells to those stimulated with IgE.
2 hr vs con Eosinophils Eosinophils were isolated from human peripheral blood using a Percoll gradient method. Eosinophils were stimulated with 50 ng/ml of Phorbol-12-myristate-13-acetate (PMA) for 2 hours at 37° C. This GeneChip compares gene expression in eosinophils activated with PMA to unactivated eosinophils.
TH1 vs TH2 CD4+ T cells were isolated from human umbilical cord blood and polarised in vitro using IL-12 and neutralising IL-4 for TH1 and polarised in vitro using IL-4 and neutralising IL-12 and interferon γ. The gene expression in TH1 cells were then compared with TH2.

The results presented in Table 1 (microarray single chip) show that PAC-1 is less broadly expressed that the other DUSPs. In particular, expression of PAC-1 is limited to T cell subsets and activated mast cells and eosinophils. DUSPs 1, 4 and 5 are expressed over a broad range of the cell types examined.

Table 2 represents all comparison GeneChips and shows fold changes of gene expression of DUSPs. The comparison GeneChip analysis shows that PAC-1 expression is 11 fold higher in RA control synoviocytes compared to OA (OA cont vs RA cont) and 15 fold higher in TNF-a stimulated RA synoviocytes compared to TNF-α stimulated OA synoviocytes (OA TNF vs RA TNF). PAC-1 expression decreases 10 fold following activation of RA synoviocytes with TNF-α (TNF RA vs cont RA) and 20 fold following IL-1β stimulation (IL-1β RA vs Control). In addition, PAC-1 expression is increased 9.6 fold following IgE activation of human mast cells (IgE vs Control MASTCELL) and 278 fold following PMA activation of human eosinophils (2 hr vs con Eosinophils).

These results demonstrate a role for PAC-1 in Rheumatoid Arthritis over Osteoarthritis and show that PAC-1 expression is decreased in activated RA synoviocytes. In addition, the results show that PAC-1 expression is increased in activated mast cells and eosinophils which indicates a role for PAC-1 in these cell types.

### Example 2

### PAC-1 Expression in Human Mast Cells Activated with IgE

Methods

CBMC culture methods: Mast cells were derived from cord blood using an established method (Ochi, H. et al. J Exp Med, 1999. 190(2): p. 267-80). Briefly, mononuclear leukocytes were isolated from cord blood using a ficoll density gradient, cells were then seeded at 2×10⁶ cells per ml of RPMI media containing 10% FBS, 1% L-glutamine, and 1% penicillan/streptomycin supplemented with 100 ng/ml stem cell factor, 10 ng/ml interleukin-10 (IL-10), and 5 ng/ml IL-6. Mast cell cultures were passaged and transferred to new flasks weekly at a concentration of 10⁶ cells per ml of media (as described above). Mast cell maturity was assessed by Toluidine blue metachromatic staining which specifically stains mast cell granules.

Mature granular mast cells were activated according to Selvan, R. S. et al J Biol Chem, 1994. 269(19): p. 13893-8. Mast cells were first primed with 4 μg/ml human IgE anti-NP for 18 hours and then activated with 5 μg/ml mouse anti-human IgE for 2 hours by crosslinking the IgE receptors. Activated and control mast cells were harvested by centrifugation and washed with PBS before lysing with Trizol reagent for RNA extraction.

Real-time PCR to monitor gene expression: cDNA of activated human mast cell RNA was made using Reverse-IT RTase Blend Kit (ABgene, UK) or Avain myeloblastosis virus Reverse Transcriptase (Promega, Madison, Wis.) according to manufacturers instructions. Oligo-p(dt)15 primer (Roche Molecular Biochemicals) was used at 1 uM in both cDNA preparation methods. Following cDNA synthesis, 1 ul of cDNA template was used for each PCR. Real-time PCR was conducted using Light Cycler-FastStart DNA Master SYBR Green I kit (Roche Molecular Biochemicals) according to manufacture's specifications using 3 mM MgCl2 and 1 uM primers.

After an initial denaturation for 1 min at 95° C., the samples were run for 40 cycles at 95° C. (15 s), 63° C. (5 s), and 72° C. (10 s). At the end of each cycle, the fluorescence was measured in a single step in channel F1. After the 40th cycle, the specimens were heated to 95° C. and cooled to 65° C. for 15 s. All heating and cooling steps were performed with a slope of 20° C./sec. The temperature was then raised to 95° C. at a rate of 0.1° C./sec and fluorescence was measured continuously (channel F1) to obtain a melting curve for the PCR products. Each gene was normalised to a housekeeping gene GAPDH before fold change was calculated (using crossing point values) to account for variations between different samples. The DUSP PCR product was confirmed by size on a 2% agarose gel.

Results

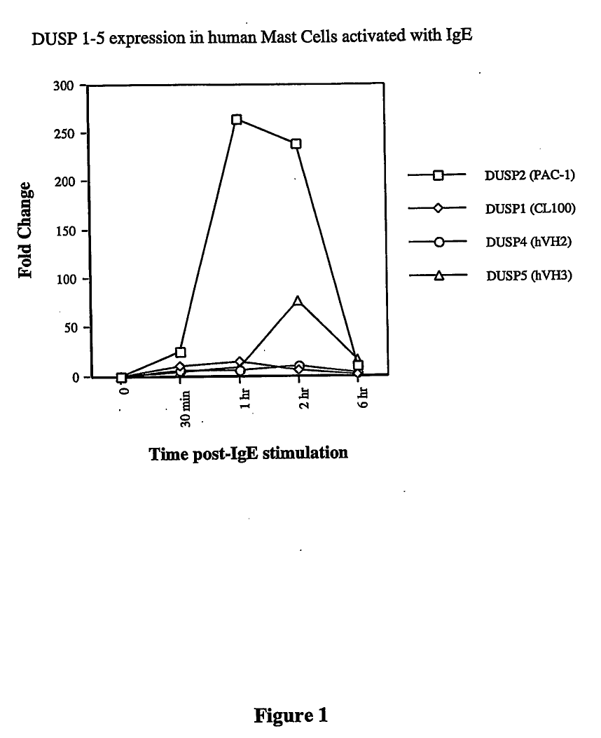

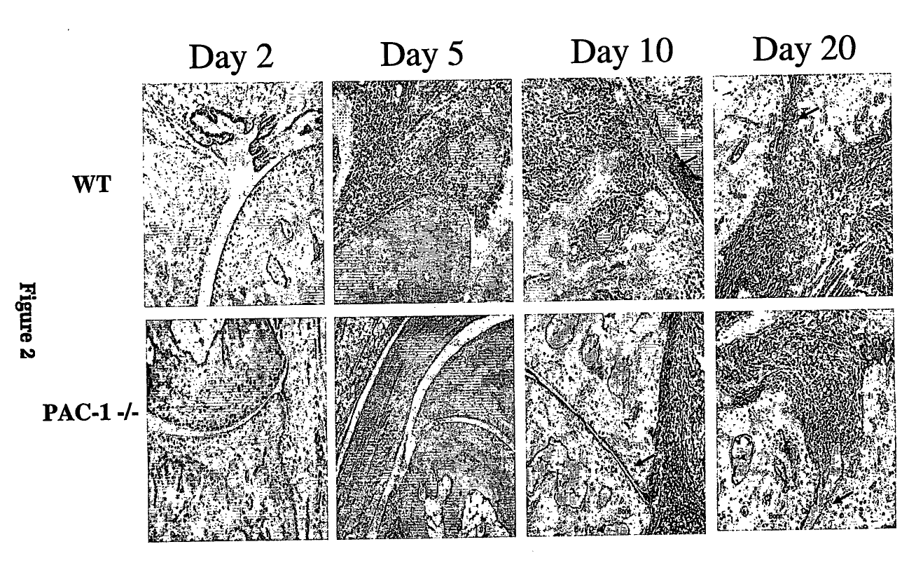

FIG. 1 shows DUSP1, DUSP2 (PAC-1), DUSP4 and DUSP5 expression in human cord blood-derived mast cells activated with IgE for 0-6 hrs (n=1). DUSP2 (PAC-1) has a higher fold change compared to other DUSPs with a peak fold change of 250 fold occurring at 1 hr and decreasing back to base levels at 6 hrs.

### Example 3

### PAC-1 Protein Expression

Immunohistochemistry analysis was performed using a polyclonal anti-PAC-1 antibody (Santa Cruz) to detect PAC-1 expression in human RA synovium (data not shown). The results showed preferential expression of the PAC-1 protein in inflamed tissue of the RA synovium. In particular, high levels of PAC-1 expression were observed in macrophages in the RA synovium.

### Example 4

### Mouse Model Studies

Methods

Recent studies using the serum of an engineered mouse model, K/B×N, have revealed that autoantibodies directed against a ubiquitously expressed antigen can selectively provoke inflammatory, hyperplastic and erosive synovitis (Kouskoff et al Cell 87:811-822, 1996). In the present study, K/B×N arthritic serum was transferred to wild type and PAC-1 deficient mice in order to investigate the functional role of PAC-1 in inflammatory arthritis.

Results

Histological analysis of ankle tissue from wild type (WT) and PAC-1 deficient (PAC-1 −/−) mice injected with the arthritic serum (K/B×N serum) showed that PAC-1 deficient mice did not display cell infiltration or bone destruction as seen in the wild type (WT) mice (FIG. 2).

Measurement of ankle thickness and clinical scores of wild type (WT) and PAC-1 deficient (PAC-1 −/−) mice injected with the arthritic serum (K/B×N serum) also showed that PAC-1 deficient mice did not develop symptoms of inflammatory arthritis as seen with wild type mice.

The PAC-1 −/− mice used in this study were backcrossed to a B6/C57 background (total of 9 backcrosses) and the results described above were reproduced. This confirms that the effects observed in PAC-1 deficient mice are due to the absence of PAC-1 rather than any strain difference between the PAC-1 deficient mice and the wild-type control mouse.

### Example 5

### Expression of PAC-1 Substrates in PAC-1 Deficient Mice

Standard Western Blot methodology was performed to analyse expression levels of the PAC-1 substrates p38 and ERK in PAC-1 deficient mice. In particular, protein samples from activated macrophages derived from PAC-1 deficient and wild-type mice were probed with anti-p38 and anti-ERK antibodies.

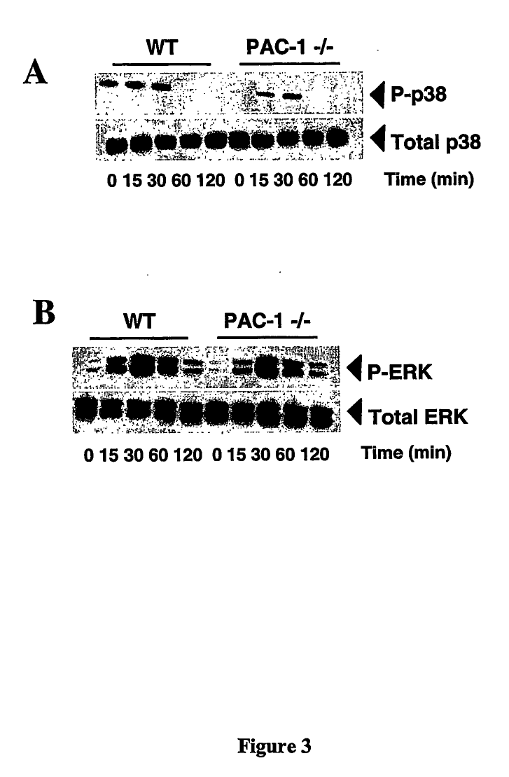

FIG. 3(A) shows significantly decreased expression of p38 in macrophages derived from PAC-1 deficient mice.

FIG. 3(B) shows a slight decrease in expression of ERK in macrophages derived from PAC-1 deficient mice.

These results cast doubt on previous reports that indicate that PAC-1 is only a negative regulator of MAP-kinases

### Example 6

### Production of Inflammatory Mediators in PAC-1 Deficient Mouse Macrophages

Experiments were performed to analyse the production of inflammatory mediators (nitrous oxide (NO), PGE2, nitrous oxide synthase (NOS-2), COX-2, IL-6 and TNF-α) in stimulated macrophages derived from PAC-1 deficient mice.

Methods

C57/136 and PAC-1 −/− mice were given an i.p. injection with 2 ml of 3% thioglycollate 4 days prior to sacrifice. Peritoneal macrophages were collected by lavaging the peritoneal cavity with 10 ml of sterile RPMI 1640 medium. The peritoneal fluid was carefully aspirated to avoid hemorrhage and kept at 4° C. to prevent the adhesion of the macrophages to the plastic. The cells were collected by centrifugation, washed, and then suspended in RPMI 1640 medium supplemented with 10% of heat-inactivated FCS. The cells were purified by adherence to tissue culture plates for 2 h Peritoneal macrophages at 2×10⁶ cells/ml were treated with 1 μg/ml LPS, 10 ng/ml IFNγ.

Results

After 18 h, N) production was determined spectrophotometrically by the accumulation of nitrite and nitrate in the medium using Griess reagent. PGE2 levels were determined in the culture medium using a specific enzyme immunoassay system, following the indications of the manufacturer (Cayman). NOS-2 and COX-2 protein expression were analyzed by western blotting. Equal amounts of protein (15 μg/lane) were loaded and electrophoresed on a 12% SDS-polyacrylamide gel. After the fractionated protein was blotted onto a nitrocellulose membrane, the membrane was incubated overnight in blocking buffer (5% nonfat dry milk, 10 mM Tris, pH 7.5, 100 mM NaCl, 0.1% Tween 20) and then treated with a goat polyclonal COX-2 antibody (Santa Cruz Lab, Calif.) for 1 h. After washing, the membrane was incubated with a horseradish peroxidase-conjugated goat IgG antibody. To detect NOS-2 the membrane was treated with a rabbit polyclonal iNOS antibody (Santa Cruz Lab, Calif.). The immunoreactive protein was detected with a chemiluminescent system (ECL kit, Amersham). Standard ELISA techniques were used to detect levels of TNF-a and IL-6.

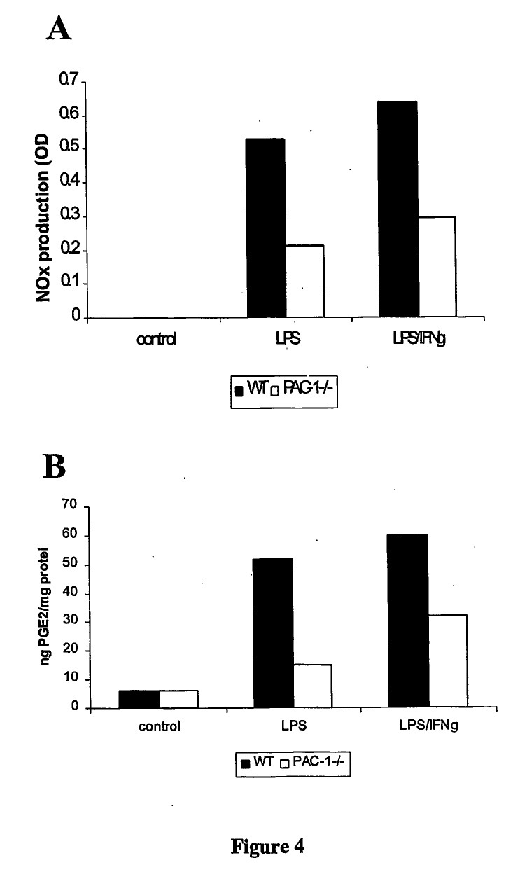

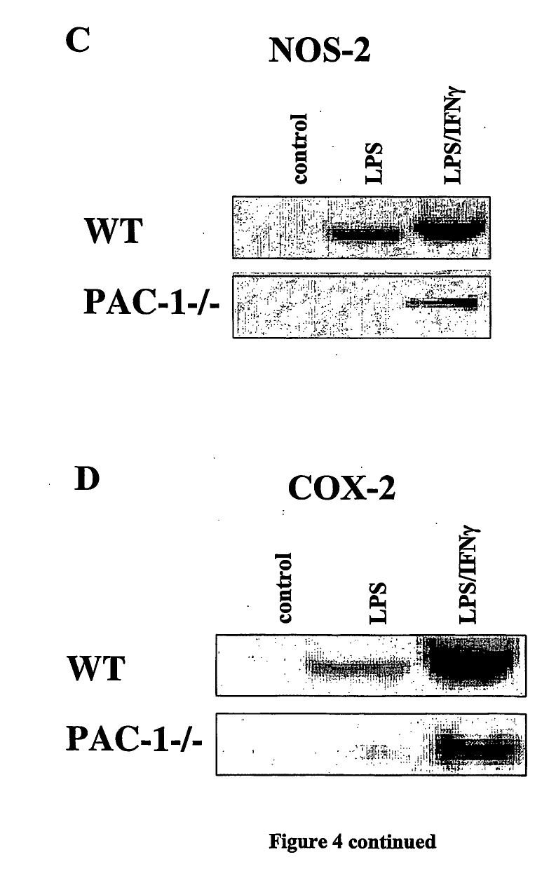

FIG. 4(A) shows that the production of NO is reduced in stimulated macrophages derived from PAC-1 deficient mice compared to stimulated macrophages derived from wild type mice.

FIG. 4(B) shows that the production of PGE2 is reduced in stimulated macrophages derived from PAC-1 deficient mice compared to stimulated macrophages derived from wild type mice.

FIG. 4(C) shows that the production of NOS-2 is reduced in stimulated macrophages derived from PAC-1 deficient mice compared to stimulated macrophages derived from wild type mice.

FIG. 4(D) shows that the production of COX-2 is reduced in stimulated macrophages derived from PAC-1 deficient mice compared to stimulated macrophages derived from wild type mice.

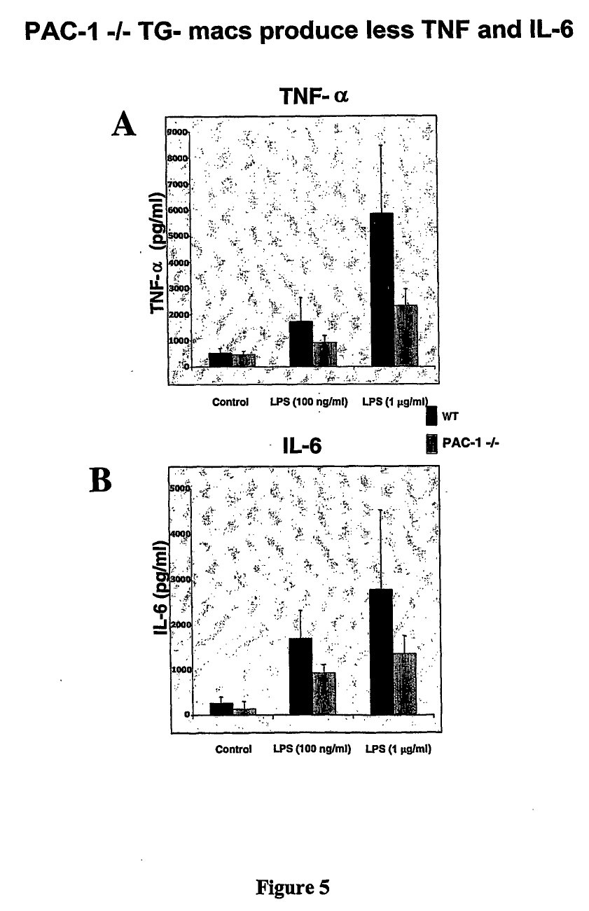

FIG. 5(A) shows that the production of TNF-α is reduced in stimulated macrophages derived from PAC-1 deficient mice compared to stimulated macrophages derived from wild type mice.

FIG. 5(B) shows that the production of IL-6 is reduced in stimulated macrophages derived from PAC-1 deficient mice compared to stimulated macrophages derived from wild type mice.

### Example 7

### Microarray Analysis of Gene Expression in PAC-1 Deficient Mouse Macrophages

Gene chip expression analysis of macrophages derived from PAC-1 deficient and wild type mice was performed using methodology essentially as described in Example 1.

Genes that showed increased expression in macrophages derived from PAC-1 deficient mice when compared with macrophages derived from wild-type mice include TYROBP, CD68, CD44 and RAB4a.

Genes that showed decreased expression in macrophages derived from PAC-1 deficient mice when compared with macrophages derived from wild-type mice include interleukin 1β, CXCL2/MIP2a, CXCL1/GRO1, IL-6 and COX2.

This gene chip analysis confirms that there is a reduced expression of inflammatory mediators in stimulated macrophages derived from PAC-1 deficient mice.

Interestingly, there was no observable increase in expression of other members of the DUSP family in PAC-1 deficient mice.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

TABLE 1

| | | | |
|---|---|---|---|
| DUSP1 | DUSP2 | DUSP4 | DUSP5 |
| (CL100) | (PAC-1) | (hVH2) | (hVH3) |

version 4

| | | | | |
|---|---|---|---|---|
| OA control | 538.3 | 0 | 101.3 | 83 |
| OA TNF | 541.1 | 0 | 0 | 395.2 |
| RA control | 239.6 | 0 | 0 | 165.9 |
| RA IL-1 | 420.9 | 0 | 0 | 763.4 |
| RA TNF | 227.6 | 0 | 0 | 667.1 |
| HMC1 | 55.3 | 0 | 1295.9 | 50.3 |
| alpha4beta7 | 1050.1 | 385.9 | 13.8 | 95.3 |
| BSCM Control | 41.6 | 0 | 0 | 66.9 |
| BSCM IL4 | 33.9 | 0 | 0 | 56.9 |
| BSCM IL13 | 32.2 | 0 | 0 | 67.5 |
| RA cont | 148.5 | 0 | 0 | 184.5 |
| RA IL-1 | 333.7 | 0 | 0 | 902.2 |
| RA TNF | 163.2 | 0 | 0 | 469.5 |
| CLA | 718.1 | 0 | 184 | 65.6 |
| RA 80 Control | 125.5 | 0 | 41.3 | 48.3 |
| RA 80 IL4 | 202.2 | 0 | 106.7 | 136.5 |
| IL 1b RA | 309.8 | 0 | 0 | 491.3 |
| MC control | −0.4 | 0 | 93.9 | 32.4 |
| MC anti-IgE Wk6 | 49.1 | 490.2 | 315.4 | 114.4 |

version 5

| | | | | |
|---|---|---|---|---|
| alpha4beta7 | 1440.3 | 297.4 | 0 | 129.2 |
| CLA | 833.2 | 0 | 281.7 | 104.8 |
| NHBE 18hr Control | 1792.0 | 0 | 322.6 | 208.7 |
| NHBE 18hr IL13 | 1509.1 | 0 | 262.4 | 157.5 |
| NHBE 18hr IL4 | 1381.0 | 0 | 260.8 | 153 |
| CCR7+ | 479 | 0 | 0 | 163.3 |
| CCR7− | 490.8 | 356.4 | 0 | 165.8 |
| CD57+ | 867.8 | 304.8 | 0 | 227 |
| CD57− | 847.5 | 0 | 0 | 225.8 |
| CD8+ CCR7− RO+ | 4133.8 | 1713.1 | 0 | 169.4 |
| CD8+ CCR7− RO− | 2890.7 | 1413.5 | 0 | 189.2 |
| TH1 human | 58.8 | 378.6 | 996.3 | 2877 |
| TH2 human | 46.7 | 418.9 | 1598.5 | 370.4 |
| Control Eosinophils | 1339 | 0 | 0 | 76.2 |
| 2hr Eosinophils | 7609.7 | 609.6 | 0 | 318.7 |
| Control | 166.2 | 0 | 160.5 | 120.6 |
| MASTCELL | | | | |
| IgE MASTCELL | 374.2 | 109.7 | 530.9 | 408 |
| Week4 Mastcell | 587 | 118.6 | 96.7 | 49 |
| Week9 Mastcell | 281.5 | 0 | 108.1 | 53.9 |

TABLE 2

| | DUSP1 | DUSP2 | DUSP4 | DUSP5 |
|---|---|---|---|---|
| | (CL100) | (PAC-1) | (hVH2) | (hVH3) |
| OA cont vs RA cont | −0.25 | −11.2 | 4.4 | −2.3 |
| OA TNF vs OA control | 0.3 | 1.9 | −2.9 | 4.4 |
| OA TNF vs RA TNF | −1.2 | −15.1 | −1.8 | −1.1 |
| RA IL-1 vs RA cont | 1.8 | 1.8 | −1.3 | 4.6 |
| RA TNF vs RA IL-1 | −1.8 | −1.2 | −1.3 | −1.1 |
| BSMC IL-13 vs cont | −1.1 | 2.5 | 1.8 | 1.7 |
| CLA vs a4b7 | 1.4 | −1.1 | 11.6 | 1.0 |
| NHBE IL13 vs cont | 1.2 | 2.4 | 1.1 | 1.5 |
| NHBE 18hr IL4 vs Control | 1.3 | 2.2 | 1.3 | 1.4 |
| TNF RA vs cont RA | 6.7 | −10.2 | −1.35 | 18.4 |
| RA IL-1 vs RA cont | 2.2 | −2.3 | −1.7 | 4.9 |
| RA IL4 vs RA cont | 1.6 | 1.0 | −2.6 | 2.7 |
| II1b RA vs Control | 24.6 | −20.7 | −1.6 | 38.8 |
| MC anti-IgE Wk6 vs cont | 5.8 | 6.6 | 3.4 | 3.5 |
| BSCM IL4 vs BSMC Control | 1.6 | −1.1 | 1.9 | −1.0 |
| BSCM IL13 vs BSMC | −1.3 | −2.1 | −1.3 | 1.1 |
| Control | | | | |
| CCR7+ vs CCR7− | 0.2 | 1.4 | 2.4 | 1.0 |
| CD57+ vs CD57− | 1.1 | 1.2 | 2.4 | 1.2 |
| CD8+ CCR7− RO vs CD8+ | −0.4 | 1.2 | 1.8 | 1.1 |
| CCR7− RO+ | | | | |
| Wk9 vs Wk4 Mastcell | −2.5 | 10.0 | 1.6 | 1.1 |
| IgE vs Control MASTCELL | 0.9 | 9.6 | 1.9 | 4.1 |
| 2hr VS con Eosinophils | 3.3 | 278.2 | 18.6 | 2.6 |
| TH2 human VS TH1human | 0.0 | 1.2 | 1.4 | 7.6 |

1. A method of screening for a compound that suppresses or reduces inflammation, the method comprising determining the activity of PAC-1 in the presence and absence of a candidate compound, wherein altered PAC-1 activity in the presence of the compound indicates that the compound suppresses or reduces inflammation.

2. A method as claim in claim 1 wherein reduced PAC-1 activity in the presence of the compound indicates that the compound suppresses or reduces inflammation.

3. A method as claimed in claim 1 wherein PAC-1 activity is determined by measuring the phosphatase activity of PAC-1.

4. A method of screening for a compound that suppresses or reduces inflammation, the method comprising determining the expression levels of PAC-1 in the presence and absence of a candidate compound, wherein altered PAC-1 expression in the presence of the compound indicates that the compound suppresses or reduces inflammation.

5. A method as claimed in claim 4 wherein reduced PAC-1 expression in the presence of the compound indicates that the compound suppresses or reduces inflammation.

6. A method as claimed in claim 4 wherein the method involves exposing a translation system capable of expressing PAC-1 to a candidate compound and comparing the levels of expression of PAC-1 in the presence of the compound to the levels achieved under similar conditions but in the absence of the compound.

7. A method as claimed in claim 6 wherein the translation system is a cell-free translation system.

8. A method as claimed in claim 6 wherein the translation system comprises eukaryotic or prokaryotic cells.

9. A method of screening for a compound that suppresses or reduces inflammation, the method comprising determining the ability of a candidate compound to modulate the binding of PAC-1 to a PAC-1 substrate, wherein an altered level of binding of PAC-1 to the substrate in the presence of the compound indicates that the compound suppresses or reduces inflammation.

10. A method as claimed in claim 9 wherein a reduced level of binding of PAC-1 to the substrate in the presence of the compound indicates that the compound suppresses or reduces inflammation.

11. A method as claimed in claim 9 wherein the PAC-1 substrate is selected from the group consisting of ERK, p38 and JNK.

12. A method of screening for a compound that promotes an immune response, the method comprising determining the activity of PAC-1 in the presence and absence of a candidate compound, wherein altered PAC-1 activity in the presence of the compound indicates that the compound promotes an immune response.

13. A method as claimed in claim 12 wherein enhanced or increased PAC-1 activity in the presence of the compound indicates that the compound promotes an immune response.

14. A method of screening for a compound that promotes an immune response, the method comprising determining the expression levels of PAC-1 in the presence and absence of a candidate compound, wherein altered PAC-1 expression in the presence of the compound indicates that the compound promotes an immune response.

15. A method as claimed in claim 14 wherein enhanced or increased PAC-1 expression in the presence of the compound indicates that the compound promotes an immune response.

16. A method of screening for a compound that promotes an immune response, the method comprising determining the ability of a candidate compound to modulate the binding of PAC-1 to a PAC-1 substrate, wherein an altered level of binding of PAC-1 to the substrate in the presence of the compound indicates that the compound promotes an immune response.

17. A method as claimed in claim 16 wherein an enhanced or increased level of binding of PAC-1 to the substrate indicates that the compound promotes an immune response.

18. A method as claimed in claim 1 wherein the candidate compound is selected from the group consisting of a peptide, a PAC-1 dominant-negative mutant, an antibody directed against PAC-1, a small organic molecule, an antisense compounds directed against PAC-1-encoding mRNA, an anti-PAC-1 catalytic molecule, dsRNA or a small interfering RNA (RNAi) molecule that targets PAC-1 expression.

19-35. (canceled)
